SEQUENCE LISTING (8 sequences)

SEQ ID NO: 1 - protein, 164 aa, Bacteriophage T4
MNIFEMLRIDEGLRLKIYKDTEGYYTIGIGHLLTKSPSLNAAKSELDKAIGRNCNGVITKDEAEKLFNQDVDAAVRGILRNAKLKPVYDSLDAVRRCALINMVFQMGETGVAGFTNSLRMLQQKRWDEAAVNLAKSRWYNQTPNRAKRVITTFRTGTWDAYKNL

SEQ ID NO: 2 - protein, 130 aa, Homo sapiens
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 3 - DNA, 1487 bp, Homo sapiens
ctagcactctgacctagcagtcaacatgaaggctctcattgttctggggcttgtcctcctttctgttacggtccagggcaaggtctttgaaaggtgtgagttggccagaactctgaaaagattgggaatggatggctacaggggaatcagcctagcaaactggatgtgtttggccaaatgggagagtggttacaacacacgagctacaaactacaatgctggagacagaagcactgattatgggatatttcagatcaatagccgctactggtgtaatgatggcaaaaccccaggagcagttaatgcctgtcatttatcctgcagtgctttgctgcaagataacatcgctgatgctgtagcttgtgcaaagagggttgtccgtgatccacaaggcattagagcatgggtggcatggagaaatcgttgtcaaaacagagatgtccgtcagtatgttcaaggttgtggagtgtaactccagaattttccttcttcagctcattttgtctctctcacattaagggagtaggaattaagtgaaaggtcacactaccattatttccccttcaaacaaataatatttttacagaagcaggagcaaaatatggcctttcttctaagagatataatgttcactaatgtggttattttacattaagcctacaacatttttcagtttgcaaatagaactaatactggtgaaaatttacctaaaaccttggttatcaaatacatctccagtacattccgttcttttttttttttgagacagtctcgctctgtcgcccaggctggagtgcagtggcgcaatctcggctcactgcaacctccacctcccgggttcacgccattctcctgcctcagcctcccgagtagctgggattacgggcgcccgccaccacgcccggctaattttttgtatttttagtagagacagggtttcaccgtgttagccaggatggtctcgatctcctgaccttgtgatccacccacctcggcctcccaaagtgctgggattacaggcgtgagccactgcgcccggccacattcagttcttatcaaagaaataacccagacttaatcttgaatgatacgattatgcccaatattaagtaaaaaatataagaaaaggttatcttaaatagatcttaggcaaaataccagctgatgaaggcatctgatgccttcatctgttcagtcatctccaaaaacagtaaaaataaccactttttgttgggcaatatgaaatttttaaaggagtagaataccaaatgatagaaacagactgcctgaattgagaattttgatttcttaaagtgtgtttctttctaaattgctgttccttaatttgattaatttaattcatgtattatgattaaatctgaggcagatgagcttacaagtattgaaataattactaattaatcacaaatgtgaagttatgcatgatgtaaaaaatacaaacattctaattaaaggctttgcaacac

SEQ ID NO: 4 - protein, 149 aa, Artificial
  Synthetic construct
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGVAAAAAAAEEEEEEHHHHHH

SEQ ID NO: 5 - DNA, 35 bp, Artificial
  Synthetic primer
tcaccacccatgggaaaggtctttgaaaggtgtga

SEQ ID NO: 6 - DNA, 48 bp, Artificial
  Synthetic primer
agctacgaagcttagtgatggtgatggtgatgcactccacaaccttga

SEQ ID NO: 7 - DNA, 73 bp, Artificial
  Synthetic primer
agctacgaagcttagtgatggtgatggtgatgtgcggcagctgccgcggcagccactccacaaccttgaacat

SEQ ID NO: 8 - DNA, 91 bp, Artificial
  Synthetic primer
agctacgaagcttagtgatggtgatggtgatgctcttcctcttcctcttctgcggcagctgccgcggcagccactccacaaccttgaacat

(19) United States
(12) Patent Application Publication
(10) Pub. No.: US 2008/0118489 A1
(43) Pub. Date: May 22, 2008

(54) Charge-Modified Lysozyme Antimicrobial Compositions, Surfactants, and Methods for Infections and Cystic Fibrosis

(75) Inventors: Gerard C. L. Wong, Urbana, IL (US); Wujing Xian, Urbana, IL (US); Lori K. Sanders, Urbana, IL (US); Kirstin R. Purdy, La Jolla, CA (US)

Correspondence Address: GREENLEE WINNER AND SULLIVAN P C, 4875 PEARL EAST CIRCLE, SUITE 200, BOULDER, CO, 80301, US

(73) Assignee: THE BOARD OF TRUSTEES OF THE UNIVERSITY OF ILLINOIS, Urbana, IL (US)

(21) Appl. No.: 11/551,313

(22) Filed: Oct. 20, 2006

Related U.S. Application Data
(60) Provisional application No. 60/729,376, filed on Oct. 21, 2005.

(51) Int. Cl. A61K 38/46; C07H 21/04; C12N 9/14

(52) U.S. Cl. 424/94.6; 435/195; 536/23.2

(57) ABSTRACT

The invention comprises charge modified antimicrobials, including charge modified lysozymes, as well as compositions and methods for potentiating antimicrobial activity by modifying a net charge level of the antimicrobial. Also provided is a method of treating microbial infections, including infections associated with cystic fibrosis, comprising administering or co-administering a compound of the invention. The invention provides compositions and methods of potentiating antibiotic treatment by administration of an at least partially cationic or positively-charged surfactant composition. Cationic lipid compositions including DOTAP:DOPE formulations are disclosed.

### CROSS-REFERENCES TO RELATED APPLICATIONS

This application claims the benefit under 35 USC 119(e) of U.S. Provisional Patent Application Ser. No. 60/729,376 filed Oct. 21, 2005, which is incorporated by reference in its entirity.

### STATEMENT ON FEDERALLY SPONSORED RESEARCH OR DEVELOPMENT

This invention was made with government support under NSF Grant DMR04-09769 and NIH Grant PHS-1R21-DK068431A awarded by the National Science Foundation and the National Institutes of Health, respectively. The government has certain rights in the invention.

### BACKGROUND OF THE INVENTION

Many antimicrobial proteins are cationic amphiphiles that can interact strongly with the anionic surfaces of microbes, thereby facilitating destabilization of microbial walls and membranes. These binding events are largely electrostatic interactions and are therefore dependent on the ionic environment. Disease states that are characterized by a large concentration of charged polyelectrolytes can present special challenges to effective administration of antimicrobials because these polyelectrolytes can sequester antimicrobials. Such sequestration can limit the efficacy of antimicrobials, as well as other cationic proteins (e.g., antibiotics). An example of one disease state characterized by a large concentration of polyelectrolytes is cystic fibrosis (CF).

Cystic fibrosis is the most common fatal, inherited disease in the United States. It is a genetic disorder resulting from the inheritance of a defective autosomal recessive gene. The average life expectancy of the 30,000 CF patients currently alive in the U.S. is under 30 years. The gene responsible for CF codes for the cystic fibrosis transmembrane conductance regulator (CFTR), a cyclic AMP regulated Cl⁻ ion channel found in the apical membranes of secretory epithelial cells. Mutations in CFTR disrupt epithelial ion transport and can lead to thick airway secretions, respiratory failure, as well as a range of other defects. Although CF is a systemic disease affecting a range of epithelial tissues, the major cause of mortality is lung disease associated with the accumulation of viscous mucus in pulmonary airways. Progressive destruction of the lung parenchyma and respiratory failure are attributed to persistent bacterial infections and the accumulation of viscous, infected mucus in pulmonary airways. There is, at present, no cure for the disease.

Although there has been much progress in gene therapy, complexities in the biology of the diseased lung still pose significant problems. Moreover, there is no technology to reconstitute precisely the normal expression of the CF gene, since CFTR is expressed in cells throughout the superficial epithelia as well as cells in the submucosal glands. Current treatment involves recombinant human Dnase I (rhDNase) aerosol combined with antibiotics to control infections. The enzyme rhDNase (Pulmozyme®), cuts entangled DNA molecules in CF mucus and reduces the viscosity of the mucus, thus reducing respiratory distress. However, clinically observed improvement is often moderate at best, and does not act against the infections that ultimately kill the patient. In addition, clinical evidence suggests that treatment with rhDNase is effective only to certain groups. Pulmozyme® treatment is also expensive (on the order of about $1000 per month). In fact, out-patient treatment by rhDNase is often more expensive than in-patient treatment. In addition, it is unclear whether enough CFTR genes can be introduced into enough epithelial cells to sufficiently impact the disease before the immune system or other mitigating factors interfere with vector delivery. There is a need for parallel or independent therapeutic strategies, including regimes to improve antimicrobial activity, to address infections that ultimately result in death of the CF patient.

One of the contributing factors to the occurrence of long-term infections in CF is the inactivation of native airway defense. The inflammatory response to infections leads to the deposition of high concentrations of negatively charged polymers in the airways, including cytoskeletal proteins such as F-actin, DNA, and other cellular debris within the viscous mucus of the pulmonary airways. These negatively charged polymers can bind to and sequester naturally-occurring positively-charged antimicrobial and antibacterial proteins or other introduced pharmaceutical agents such as antibiotics, so that they can no longer fulfill their normal or desired antimicrobial function, contributing to patient debilitation or death from infection.

Other techniques have attempted to improve antimicrobial activity by using charged polymers to dissociate DNA and actin bundles in CF mucus (Tang et al., 2005). That work, however, does not involve modification of antibacterial proteins so as to obtain non-stick versions of the proteins, nor does it involve pacifying the charged surfaces with surfactants. The present invention is associated with substantial recovery of activity; non-stick antibacterial proteins are engineered to reduce sequestration and to demonstrate activity.

The present invention provides several solutions, for example, compositions and methods to improve antimicrobial activity by reducing sequestration of charged antimicrobial proteins by the oppositely charged polymers in the airways. The invention provides methods to engineer non-stick, charge-reduced versions of antibacterial proteins by analyzing and understanding the underlying electrostatic binding process. These charge-reduced antibacterial proteins can be utilized within a suite of aerosol-deliverable therapeutics that can remain active in the unusual electrostatic environment of the airway surface liquid (ASL), and at least partially restore antimicrobial function in the CF airway. By the application of this strategy, compositions and methods of the present invention can also be utilized in other infected biological systems, including for example chronic infections with a prolonged inflammatory response. The present invention also provides compositions and methods for improving efficacy of antibiotics; this can be accomplished by pacifying the charged surfaces that normally function to sequester antibiotics. Further aspects of the invention are also described.

### SUMMARY OF THE INVENTION

The following abbreviations are applicable. Cystic fibrosis (CF); Airway Surface Liquid (ASL); synchrotron small angle x-ray scattering (SAXS); cystic fibrosis transmembrane conductance regulator (CFTR); Pseudomonas aeruginosa (PA01); Charge-Coupled Device (CCD); 1,2-Dioleoyl-3-Trimethylammonium-Propane (DOTAP); 1,2-Dioleoyl-sn-Glycero-3-Phosphoethanolamine (DOPE).

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

The invention comprises charge-modified antimicrobials and methods of potentiating antimicrobials by modifying the charge of an antimicrobial. In an embodiment, the charge-modified antimicrobial is a derivative of a reference antimicrobial wherein the derivative has a reduction of a net charge relative to the reference antimicrobial. The reduction in net charge level can be obtained by modifying one or more of the amino-acids of the reference antimicrobial so as to reduce the net charge of the derivative relative to the reference antimicrobial. The modification can be by amino acid deletion, substitution and/or alteration. A single amino acid substitution can effect a net 2 reduction in charge (e.g. substituting lysine or arginine with glutamic acid). Substitutions involving aspartic acid can also effect a net 2 reduction in charge. An amino acid deletion can effect a net 1 reduction in charge. Modification techniques as known in the art, including site-directed mutagenesis, can be used to modify one or more amino acids, thereby reducing the charge of a reference antimicrobial so long as measurable antimicrobial activity remains.

The reference antimicrobial can include any protein, peptide, or functional fragments thereof that kill and/or inhibit growth of microbes, including bacteria. Antimicrobials can be lysozyme, β-defensins, lactoferrin, and functional fragments thereof. In one embodiment the antimicrobial is a lysozyme obtained from any lysozyme-producing source. The antimicrobial source can be mammalian, bacterial or of other species origin. In an embodiment the antimicrobial is a lysozyme. In an embodiment the antimicrobial origin is mammalian, and more preferably human in origin. Antimicrobials such as human lysozyme can be expressed and isolated from recombinant systems, e.g., using bacteria or other expression hosts as known in the art. In a particular embodiment, the invention provides a charge-modified human lysozyme.

The invention comprises charge-modified antimicrobials, wherein the reduction of net charge is 1 or greater. In an embodiment, the reduction of net charge is selected from the group consisting of about 2, about 3, about 4, about 5, about 6, about 7, and about 8. In an embodiment the reduction of net charge is at least about 2. In an embodiment the reduction of net charge is at least about 4. In an embodiment the reduction in net charge is such that the charge-modified antimicrobial has a measurable improvement in antimicrobial activity compared to the reference antimicrobial when administered to a patient suffering from a disease. Preferably, the improvement is at least 10%, at least 50%, at least about 60%, at least about 70%, at least about 80%, or at least about 90% relative to the reference antimicrobial. The reference antimicrobial can have a relative antimicrobial activity selected from the group consisting of at least about 50%, at least about 60%, at least about 70%, at least about 80%, and at least about 90% in comparison with a reference antimicrobial activity of the reference lysozyme protein.

The invention encompasses methods for potentiating the antimicrobial activity of a protein, a peptide, or functional fragment thereof, by modifying the net charge level of the protein, the peptide, or the functional fragment thereof. In one embodiment the method is for modifying the net charge level of lysozyme. In an embodiment the lysozyme's net charge level is modified to a less positive net charge level.

The invention encompasses methods of treating a microbial infection, comprising administering to a patient in need of the composition any of the compositions of the present invention. In an embodiment, the administration occurs by aerosol delivery. In an embodiment the administration is to the air passage of a patient, including administration to the upper respiratory tract region. In an embodiment the patient is a cystic fibrosis patient. In addition to the context of cystic fibrosis, compounds and methods of the invention are suitable as therapies in chronic infection conditions including such with a prolonged inflammatory response. Furthermore, charge-modified lysozymes in particular are applicable in compositions and methods relating to artificial tears, artificial saliva, and infant formulas, milks, and supplements thereto.

The invention encompasses methods of generating a non-stick, charge-modified form of an antimicrobial protein comprising: (a) providing a candidate antimicrobial protein or sequence information corresponding to nucleic acids or amino acids thereof; (b) developing at least one charge-modified version of said candidate antimicrobial protein; (c) screening said charge-modified version for antimicrobial activity; and (d) selecting an active charge-modified version; thereby generating said non-stick, charge-modified form of an antimicrobial protein. In one embodiment the charge-modified version is developed by substituting at least one positively-charged amino acid with at least one negatively-charged amino acid.

The invention provides surfactant compositions. The invention encompasses methods of potentiating an antibiotic treatment, comprising the steps of (a) administering a surfactant composition; and (b) administering the antibiotic to a patient in need of treatment. In an embodiment, the surfactant composition is administered before antibiotic treatment. In an embodiment, the surfactant and antibiotics are administered substantially simultaneously. The surfactant composition can comprise natural (e.g. lipids) and/or artificial (e.g. artificially synthesized) components and/or amphiphilic molecules. The surfactant composition can comprise a cationic lipid composition. The surfactant composition can comprise a combination of cationic lipid and neutral lipid. The surfactant composition can comprise essentially pure cationic lipid. The surfactant composition can comprise about 100% nominally neutral lipids. The nominally neutral lipids can have both positive and negative charges that cancel. In an embodiment, the surfactant composition is at least partially cationic.

In an embodiment, the surfactant composition is selected from the group consisting of: Didodecyldimethylammonium bromide (DDAB); Cetyltrimethylammonium bromide (CTAB); Cetyltrimethylammonium bromide (CTAB); 1-palmitoyl-2-hydroxy-sn-glycero-3-phosphocholine (PHGPC) from 20:80 to 80:20; DLTAP:DLPC, DOTAP:DOPC, and DNTAP:DNPC (dilauryl trimethyl ammonium propane: dilauryl trimethyl phosphatidylcholine, dioleoyl trimethyl ammonium propane: dioleoyl trimethyl phosphatidylcholine, and dinervonyl trimethyl ammonium propane: dinervonyl trimethyl phosphatidylcholine, respectively) from 100:0 to 10:90.

In an embodiment, the surfactant composition is selected from the group consisting of: 1,2-Diarachidonoyl-sn-Glycero-3-Phosphoethanolamine; 1,2-Didocosahexaenoyl-sn-Glycero-3-Phosphoethanolamine; 1,2-Dielaidoyl-sn-Glycero-3-Phosphoethanolamine; 1,2-Dihexanoyl-sn-Glycero-3-Phosphoethanolamine; 1,2-Dioctanoyl-sn-Glycero-3-Phosphoethanolamine; 1,2-Dihexanoyl-sn-Glycero-3-Phosphoethanolamine; 1,2-Dilauroyl-sn-Glycero-3-Phosphoethanolamine; 1,2-Dimyristoyl-sn-Glycero-3-Phosphoethanolamine; 1,2-Dipalmitoyl-sn-Glycero-3-Phosphoethanolamine; 1,2-Dipalmitoleoyl-sn-Glycero-3-Phosphoethanolamine; 1,2-Diheptadecanoyl-sn-Glycero-3-Phosphoethanolamine; 1,2-Dicapryl-sn-Glycero-3-Phosphoethanolamine; 1,2-Dimyristoyl-3-Trimethylammonium-Propane; 1,2-Dipalmitoyl-3-Trimethylammonium-Propane; 1,2-Dimyristoleoyl-sn-Glycero-3-Phosphocholine; 1,2-Dimyristelaidoyl-sn-Glycero-3-Phosphocholine; 1,2-Dipalmitoleoyl-sn-Glycero-3-Phosphocholine; 1,2-Dipalmitelaidoyl-sn-Glycero-3-Phosphocholine; 1,2-Dieicosenoyl-sn-Glycero-3-Phosphocholine; 1,2-Dierucoyl-sn-Glycero-3-Phosphocholine; 1,2-Dinervonoyl-sn-Glycero-3-Phosphocholine; 1,2-Dipetroselinoyl-sn-Glycero-3-Phosphocholine; and 1,2-Dielaidoyl-sn-Glycero-3-Phosphocholine.

In an embodiment, the surfactant composition comprises DOTAP and DOPE.

In an embodiment, a surfactant composition is provided in a formulation capable of forming a lamellar or inverse hexagonal phase with a complexing agent such as DNA. In an embodiment, a cationic lipid composition is provided. In an embodiment the surfactant composition comprises DOTAP and DOPE. In an embodiment the DOTAP:DOPE ratio is from about 100:0 to about 10:90. In a particular embodiment, this ratio is from about 70:30 to about 25:75. In an embodiment, the antibiotic is from the aminoglycoside family of antibiotics, wherein the aminoglycoside is positively-charged. In an embodiment, the antibiotic is selected from the group consisting of tobramycin, gentamycin, kanamycin, streptomycin, neomycin, amikacin, and ampramycin. In an embodiment, the antibiotic is tobramycin. In an embodiment the patient has a chronic microbial infection. In an embodiment the patient is at risk for a chronic microbial infection. In an embodiment the patient is a cystic fibrosis patient. In an embodiment, the composition is a medicament for treatment of an infection.

In an embodiment the surfactant composition is positively charged. In an embodiment, the surfactant composition is at least partially cationic. In an embodiment the surfactant composition comprises amphiphilic molecules, including but not limited to block copolymers. In an embodiment the invention comprises compositions and methods relating to amphiphilic molecules. In a particular embodiment, the amphiphilic molecules are surfactants. In a particular embodiment, the amphiphilic molecules are lipids. In a particular embodiment, the amphiphilic molecules are not surfactants or lipids. In a particular embodiment, the amphiphilic molecules are amphiphilic polymers. In a particular embodiment, the amphiphilic polymers are block copolymers. In a particular embodiment, the amphiphilic molecules are at least partially cationic.

In an embodiment the invention is a method of generating a positively-charged surfactant formulation for therapeutic use in connection with a positively-charged antimicrobial agent, wherein said therapeutic use involves an electrostatic environment with at least one anionic component, comprising: (a) identifying said at least one anionic component; (b) providing a positively charged surfactant formulation candidate; (c) adapting said formulation candidate so as to at least partially optimize one or more properties for interaction with said anionic component; (d) measuring an entropic parameter of said candidate upon binding said anionic component; and (e) selecting a formulation candidate exhibiting an entropy gain from said measuring step; thereby generating a positively-charged surfactant formulation for therapeutic use in connection with the positively-charged antimicrobial agent. In an embodiment, the properties in step (c) comprise a charge level and a structural conformation property.

The invention encompasses a method of generating a positively-charged surfactant formulation for therapeutic use in connection with a positively-charged antimicrobial agent, wherein said therapeutic use involves an electrostatic environment with at least one anionic component, comprising: (a) identifying said at least one anionic component; (b) providing a positively charged surfactant formulation candidate; (c) maximizing an entropic gain of said candidate upon binding said anionic component by optimizing one or more of charge density and surfactant curvature of said surfactant formulation candidate; and (d) selecting a formulation candidate exhibiting an entropy gain from said maximizing step; thereby generating a positively-charged surfactant formulation for therapeutic use in connection with the positively-charged antimicrobial agent.

The invention encompasses a charge-modified antimicrobial lysozyme, wherein the charge-modified lysozyme is a derivative of a reference lysozyme protein and has one or more charge decreases relative to the reference lysozyme protein.

The invention encompasses any of the charge-modified lysozymes disclosed herein, excepting a mutant T4 bacteriophage lysozyme as described herein and those other lysozymes which may be known in the art that do qualify as prior art.

In an embodiment, a composition of the invention is isolated or purified.

In an embodiment, the invention provides a method of potentiating an antibiotic treatment, comprising the steps of (a) administering an amphiphilic molecule composition; and (b) administering the antibiotic to a patient in need of treatment.

In an embodiment, the invention provides a method of generating an amphiphilic molecule formulation for therapeutic use in connection with a positively-charged antimicrobial agent, wherein said therapeutic use involves an electrostatic environment with at least one anionic component, comprising: (a) identifying said at least one anionic component; (b) providing an amphiphilic molecule formulation candidate; (c) maximizing an entropic gain of said candidate upon binding said anionic component by optimizing one or more of charge density and curvature of said candidate; and (d) selecting a formulation candidate exhibiting an entropy gain from said maximizing step; thereby generating an amphiphilic molecule formulation for therapeutic use in connection with the positively-charged antimicrobial agent.

In an embodiment, the amphiphilic molecule is at least partially cationic.

In an embodiment, the invention provides a charge-modified mammalian lysozyme comprising a first segment having of an amino acid sequence of a mammalian lysozyme, and a second segment having from about two to about ten negatively charged amino acids. In an embodiment, the second segment comprises six negatively charged amino acids. In an embodiment, the second segment comprises six glutamate residues.

In an embodiment, the charge-modified mammalian lysozyme further comprises a third segment of a spacer, wherein said spacer is positioned between said first segment and said second segment. In an embodiment, the spacer is a peptide comprising from about two to about ten amino acids. In an embodiment, the spacer comprises seven alanine residues. In an embodiment, the lysozyme is human. In an embodiment, the lysozyme has the amino acid sequence of SEQ ID NO:4.

In an embodiment, the invention provides a nucleic acid sequence capable of encoding a charge-modified lysozyme.

In an embodiment, the invention provides a method of treating an infection condition involving a prolonged inflammatory response, comprising administering to a patient in need the composition of the invention. In an embodiment, the infection is a chronic microbial infection.

The invention provides pharmaceutical compositions and formulations of antimicrobial compositions described herein.

### BRIEF DESCRIPTION OF THE FIGURES

FIG. 1: Schematic representation of cation induced F-actin bundles. Cations organize into density waves which twist the actin helix to optimize electrostatic contact From Angelini et al. (2003) PNAS 100, 8634-8637.

FIG. 2: Examples of supramolecular cation-anion self-assembly: (A) DNA-cationic membrane complexes (Radler et al, Science, 1997); (B) actin-cationic membrane complexes (Wong et al., Science, 2000); (C) Cation-induced formation of stacked actin networks (Wong et al., Phys. Rev. Lett., 2003).

FIG. 3: Structure of lysozyme-actin composite bundles from synchrotron x-ray diffraction. Lysozyme (light gray) is close-packed in 3-fold symmetric sites between actin filaments (dark gray).

FIG. 4: (A) Synchrotron 2-D x-ray diffraction of partially aligned lysozyme-actin bundles. (B) 1-D integrated slices along the qz and qr directions with arrows marking the actin-actin closed-packed bundling peak (1), the actin helix form factor (2), and the lysozyme-lysozyme correlation peak (3). (C) Series of diffraction data showing evolution of bundle structure as a function of NaCl concentration. Note enhanced lysozyme-actin binding (noted by arrow) as NaCl concentration is increased in the 50 mM-150 mM range. The lysozyme-actin bundling peak then disappears as the salt concentration is increased above 200 mM.

FIG. 5: (A) Schematic of microdiffraction experiment at the Advanced Photon Source. (B) Representative 1-D integrations from x-ray microdiffraction measurements from CF sputum collected from Carle Clinic, Urbana, Ill., which exhibit correlation peaks at the same q-position as those from in vitro lysozyme-actin bundles.

FIG. 6: Schematic of T4 charge-reduced lysozyme mutant (18.7 kDa) with a two site mutation, at positions 16 and 119. In the 16/119 mutant at K16E, lysine has been replaced with glutamic acid. At R119E, arginine has been replaced with glutamic acid. The resultant protein has a total charge of +5 rather than +9 found in the wild-type.

FIG. 7 (A) shows the schematic of lysozyme-actin coordination for wild type T4 lysozyme and charge-reduced lysozyme mutant. At low salt levels (0-50 mM NaCl), the charge-reduced lysozyme is stabilized at 2-fold rather than 3-fold sites. FIG. 7 (B) Synchrotron x-ray diffraction data integrated in one dimension along the qz direction of a partially aligned lysozyme-actin bundle. Diffraction data from the wild-type (+9) lysozyme-actin complex is shown in red, while the double mutant (+5) lysozyme-actin complex is shown in black. A shift in the position of the actin-actin bundling peak to lower q, as indicated by the arrows, indicates a change in lysozyme coordination in the complex.

FIG. 8 shows SAXS data at different monovalent salt levels for wild-type lysozyme-actin complexes (FIG. 8A) and 16/119 mutant lysozyme-actin complexes (FIG. 8B). The shift in the actin-actin distance indicates that the lysozyme has changed coordination. Moreover, the region of stability of such complexes for mutant lysozyme complexes (brackets, FIG. 8B) occurs at a smaller range of much lower salt levels than that for the wild-type lysozyme (brackets, FIG. 8A), which straddle physiological relevant values. This indicates that the mutant lysozymes can be unsequestered in CF airways.

FIG. 9 shows results from microequilibrioum dialysis experiments of actin-WT and actin-double mutant lysozyme (16/119). ΔC_{lysozyme} is the deviation from complete equilibrium of lysozyme as induced by the presence of actin filaments. In the absence of actin (cross hatched bars), both wild-type (dark gray, hatched) and 16/119 mutant (light gray, hatched) lysozyme is nearly fully equilibrated. For wild-type lysozyme (dark gray, solid) initially complexed with actin, the dialysis shows a much larger deviation from equilibrium than for the double mutant (light gray, solid) indicating that the the wild-type lysozyme is more strongly sequestered than the mutant lysozyme.

FIG. 10 shows results from antimicrobial microdilution assays used to determine the bactericidal activity of wild-type and the 16/119 mutant lysozymes on Pseudomonas aeruginosa (PAO1), a gram negative bacterium. Both wild-type (black trace) and 16/119 mutant (gray trace) lysozyme show increasing bactericidal activity with increasing concentrations, demonstrating that site-directed mutagenesis has not detrimentally affected the lysozyme efficacy for the charge-modified mutant.

FIG. 11 shows a bacterial killing assay for suspensions containing either tobramycin alone, or a mixture of tobramycin and 10 mg/ml mucin. Percent of live bacteria is calculated relative to the control bacterial growth with no added mucin or tobramycin. Not only does mucin enable PA01 increased bacterial growth in the absence of tobramycin, but it also inhibits the killing ability of the tobramycin until the tobramycin concentration is 20× the minimal inhibitory concentration (MIC).

FIG. 12 shows the unit cell of the structure of DNA-tobramycin composite bundles from synchrotron x-ray diffraction. DNA is hexagonally packed with spacing very near the bare diameter of DNA (about 20 Angstroms).

FIG. 13: (A) SAXS data of suspensions of F-actin (at 5 mg/ml)+DNA (at 1 mg/ml)+tobramycin, showing the appearance of the bundled DNA peak at low tobramycin concentrations and the bundled DNA and F-actin peaks at high tobramycin concentrations. (B) SAXS data of suspensions of DNA (at 3 mg/ml)+tobramycin at pH 7 showing the appearance of a bundled DNA peak at 2.6 nm⁻¹ when the ratio of tobramycin charge to DNA charge (T/D) approaches 1 (1.9 mM).

FIG. 14 shows SAXS data of suspension of DNA+tobramycin and DNA+lipid+tobramycin at different concentrations of tobramycin. The fractions listed are the ratio of tobramycin to DNA charge (T/D) or lipid to DNA charge (L/D). The lipid concentration was held fixed at 30 mM, equal to the amount required to neutralize the charge of 2.5 mg/ml DNA (L/D=1). Independent of tobramycin concentration, some of the DNA is complexed with the lipids as indicated by the indexed lamellar (FIG. 14A) or hexagonal (FIG. 14B) peaks. At low tobramycin concentrations, the DNA-tobramycin bundling peak disappears (arrows).

FIG. 15 (A) SAXS data showing that when tobramycin concentration is less than that required to neutralize DNA charge DNA is complexed with lipids. (B) For T/D approaching 1, some tobramycin complexes with DNA, but if the lipid concentration is increased to L/D=2 the tobramycin is again unsequestered by the DNA. In both cases of T/D=0.5 and T/D=0.9, tobramycin is sequestered by DNA in the absence of lipids.

FIG. 16 illustrates amino acid sequence information for wild-type bacteriophage T4 lysozyme, Accession number: 2LZM (SEQ ID NO:1) and human lysozyme, Accession number: 1LZ1 (SEQ ID NO:2).

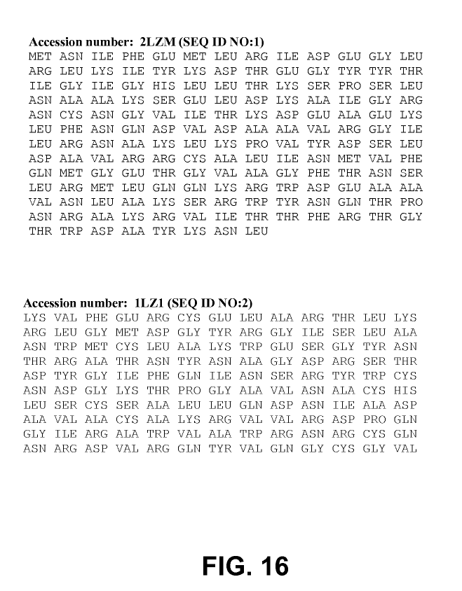

FIG. 17 illustrates two different protein engineering approaches to reduce net positive charges on a protein. A. The charge reversal approach: two positively charged amino acids are replaced by two negatively charged ones, and the net positive charge is changed from +8 to +4. B. The charge balance approach: 4 negatively charged amino acids are attached to the protein (with an optional spacer), thus reducing the net positive charge from +8 to +4.

FIG. 18 illustrates the structure in (A) of human lysozyme with a bound N-acetylglucosamine oligomer (NAG)₄. Polysaccharides in peptidoglycan, a major bacteria cell wall component, can bind to the same cleft and possibly extend towards the N-terminus of the protein. For this reason, the C-terminus, which is spatially separated from the active site, has been chosen as the preferred attachment point for charge-balancing modifications. Shown in (B) are three constructs of charge-balanced human lysozyme variants. HLYH: A six-histidine (6×His) tag is attached to the C-terminal end of human lysozyme. HLYAH: A spacer sequence of seven alanines (7×Ala) is attached to the C-terminal end of human lysozyme, followed by a 6×His tag. HLYAEH: A 7×Ala spacer is attached to the C-terminal end of human lysozyme. It is followed by a charge-balancing sequence of six negatively charged glutamates (6×Glu), and a 6×His tag.

FIG. 19 shows the DNA/mRNA sequence corresponding to human lysozyme (Accession No. NM_{—}000239).

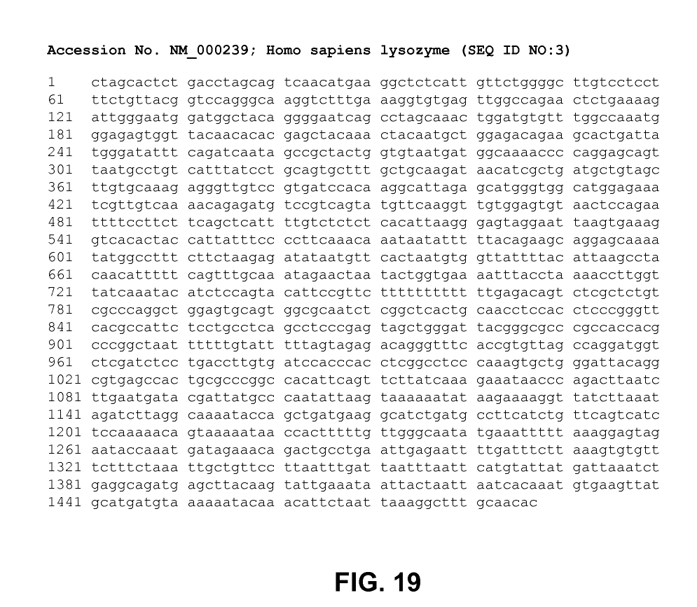

### DETAILED DESCRIPTION OF THE INVENTION

In general the terms and phrases used herein have their art-recognized meaning, which can be found by reference to standard texts, journal references and contexts known to those skilled in the art. The following definitions are provided to clarify their specific use in the context of the invention.

When used herein, the term “charge-modified” refers to an alteration in a charge level of a compound relative to that of a reference compound. A charge-modified compound can have an increased positive charge or an increased negative charge relative to the reference compound. In an embodiment, the charge-modified compound is derived from the reference compound, e.g. a native protein or variant thereof. For example, a charge-modified lysozyme is derived from a native mammalian (preferably human) lysozyme. More widely, a charge-modified lysozyme is a derivative of a reference lysozyme such as any lysozyme whether native, mutant, or other variant (preferably of mammalian origin, and more preferably of human origin), which may be relatively suboptimal with respect to a charge parameter for applications pertinent to the invention. In a preferred embodiment, a charge level is reduced from a greater net charge level to a lesser net charge level, wherein, greater and lesser can refer to a simple mathematical relationship. For example, the terminology can describe a change from an initial +9 charge level for wild-type bacteriophage T4 lysozyme to a resulting +5 charge level for a mutant T4 lysozyme. Alternatively, a resulting charge level that is a negative number is encompassed because mathematically it can have a lesser net charge level than an initial charge level of a positive number, zero, or a negative number which is greater than the resulting charge level.

When used herein, the term “non-stick” refers to a property of a compound to have a tendency not to stick to another compound or a mixture of compounds. The term is not absolute in requiring a complete absence of stickiness. In an embodiment, the term refers to an at least partially reduced ability to interact in electrostatic binding. In an embodiment, a non-stick protein has a decreased positive charge level and has a lesser susceptibility of attraction to and/or sequestration within an environment of an airway surface liquid. In a particular embodiment, the airway surface liquid is in a cystic fibrosis context and contains one or more of anionic polyelectrolytes, and includes biopolymers such as DNA, F-actin, glycoproteins such as mucin, and other cellular debris. The invention encompasses other diseases involving liquids enriched in anionic polyelectrolytes, for example lung diseases, pneumonias, and other severe infections. Any disease characterized by a large concentration of inflammatory polymers (DNA, actin, cell debris, etc.) can be treated using the compositions and methods of the present invention.

When used herein, the term “derivative” in the context of a protein refers to a mutant or variant version relative to a reference protein. For example, in an embodiment the mutant or variant has one or more of at least one changed natural amino acid (substitution), truncation or other deletion, or addition relative to a native or reference sequence. In an embodiment, the mutant or variant has a modification such as a non-natural amino acid substitution, addition, or chemical modification to an amino acid or the protein molecule as would be understood in the art. A derivative can be prepared by synthetic and/or recombinant techniques.

When used herein, the term “positively charged” indicates the presence of at least one positive charge in a molecule and therefore can be synonymous with a description of being at least partially cationic. A subset of positively charged molecules can have a net overall positive charge, for example if the total number of plus charges is greater than the total number of minus charges for a given molecule.

When used herein, the term “potentiating” refers to enhancing or improving a performance property relative to a reference level. For example, an antibiotic activity can be potentiated by administration of a positively-charged surfactant composition. In an embodiment, the surfactant administration can be before, after, or simultaneous with (co-administration) introduction of the antibiotic.

When used herein, the term “surfactant composition” is used broadly to refer to surface-active agents. The surfactant composition can be lipids, wherein the lipids can be biological, artificial (e.g. chemically synthesized) or partially biological and partially artificial in original. The surfactant composition can comprise nominally “neutral” lipids, comprising both positive and negative charges in amounts such that the net charge on the lipid is neutral. In an embodiment the surfactant composition is at least partially cationic. In an embodiment the surfactant composition is positively charged. The surfactant composition can comprise mixtures of multiple lipids and/or multiple surfactants.

When used herein, the term “unsequestered” or “not sequestered” generally refers to a state of a substance being not bound or less bound to another moiety or complex. Regarding an individual substance or population of substances, the term can indicate that there is less than a complete degree of sequestration, e.g., of an individual molecule or population of molecules. The term is consistent with a state of being released or liberated, or remaining free or resistant to being bound, whether from a previously bound state or without having previously been bound.

The invention may be further understood by the following non-limiting examples.

### EXAMPLE 1

### Electrostatic Interactions of Biological Polyelectrolytes

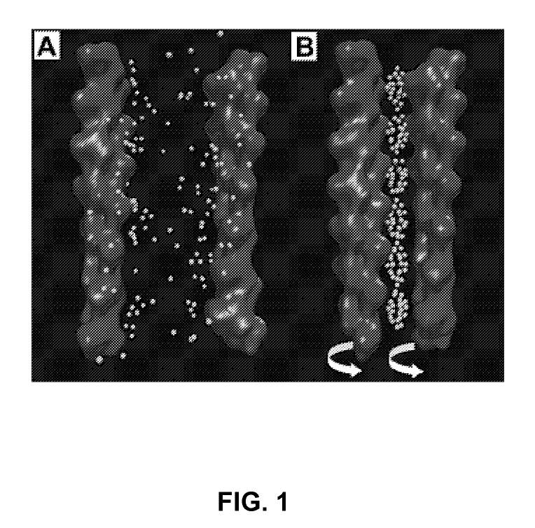

The electrostatic behavior of polyelectrolytes such as DNA and F-actin is considerably more complex than uncharged polymer fluids. In the presence of oppositely charged multivalent cations, both DNA and F-actin can overcome their mutual electrostatic repulsion and attract one another and organize into new self-assembled phases. Examples from nature include the hierarchical ordering of DNA chains via histones within chromosomes, and the high-density liquid crystalline DNA packaging by multivalent protamines in bacteria and viral capsids. We have experimentally established a microscopic mechanism for cation-mediated attraction between F-actin, and find that the cations organize into density waves that induce nanomechanical twist distortions of the actin helix (FIG. 1) in order to enhance the zipper-like charge alignment. Angelini et al. (2003).

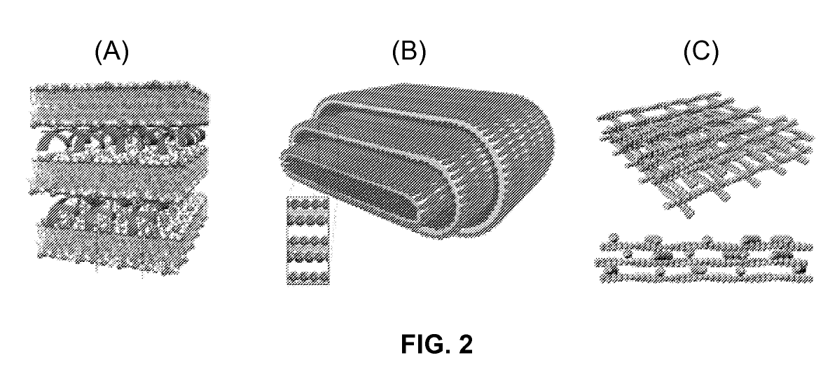

The behavior of these condensed polyelectrolyte phases becomes even richer with increasing complexity in the condensing cations. The CF airway is ‘littered’ with both anionic and cationic components that can interact electrostatically. These electrostatic complexes can be understood. For example, gene delivery systems such as DNA-cationic polymers or DNA-cationic membrane complexes have recently received extensive experimental and theoretical scrutiny. See Raedler et al. (1997); Feigner et al. (1987,1991); Gustafsson et al. (1995); Sternberg et al. (1994); Lasic et al. (1997); May et al. (1997); Bruinsma (1998); Koltover et al. (1998). A polymorphism of different structures of these DNA-membrane complexes (such as the lamellar and hexagonal phases) with different transfection efficiencies can exist, as elucidated by recent synchrotron x-ray scattering experiments (FIG. 2). The cationic molecules used in the packaging of DNA can interact with adventitious F-actin, which exhibits a similar level of complexity in its interactions with such molecules. For example, using a combination of synchrotron x-ray scattering and electron microscopy (EM) we found that F-actin can organize into a range of different well-defined liquid crystalline phases, from lamellar stacks of 2-D networks to uniaxial bundles (FIG. 2). The rules governing this form of self-assembly, which depend on entropically modulated electrostatic interactions, can even dictate the formation of new phases no longer constrained by the typical liquid crystalline geometries. For example, the addition of cationic lipids (such as those used to package DNA in non-viral gene delivery systems) to F-actin can produce osmotically stabilized layered structures that are in turn hierarchically organized into mesoscopic tubules, in a process which was recently elucidated using synchrotron x-ray diffraction and freeze fracture electron microscopy (FIG. 2). The relative stability of all of these phases is determined by their structures, which can be directly measured.

### EXAMPLE 2

### Structure of Antibacterial Peptides Electrostatically Sequestered With Biological Polyelectrolytes

Condensed bundles comprised of F-actin and of DNA occur in CF sputum (Sheils et al. 1996), since these polymers arise in the ASL when neutrophils and other cells lyse as the result of the inflammatory response. It has been suggested that cationic antibacterial polypeptides constitute at least a portion of the ligands holding these polyelectrolytes together. Weiner et al. (2003). We have examined F-actin-lysozyme complexes and determined that lysozyme close-packs into a 1-D column in between a hexagonal arrangement of F-actin filaments. More importantly, the F-actin-lysozyme binding is enhanced at elevated NaCl and KCl concentrations. This is consistent with experimental results in which antibacterial activity is increased as the salt level of the ASL was artificially lowered using an osmolyte. Zabner et al. (2000). By directly measuring the structure and relative stability of these complexes across a wide range of buffer conditions (salt, pH, temperature) using synchrotron x-ray diffraction, and using the data synergistically with computer simulations, rational design dissolution strategies are obtained. From the present invention, one skilled in the art can tailor-design, using biophysical considerations, charge-reduced mutant antibacterial peptides that bind to F-actin and DNA much less than endogenous antibacterial peptides, and therefore, can be active in the electrostatic environment of the airway. Biophysical tools such as synchrotron x-ray diffraction and computer simulations are used to elucidate the nature of the binding and the structure of the resultant complexes (e.g. polymer sequestration of native versus charge reduced antimicrobials).

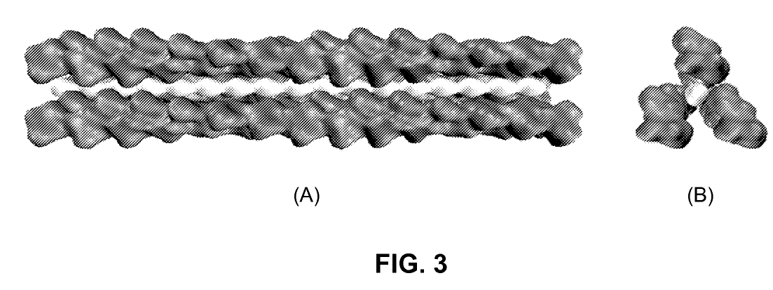

In order to assess the relative stabilities of the different polyelectrolyte complexes that occur in CF mucus, it is necessary to know their structures. Actin can condense into bundles with model cationic ligands. We have solved the structure of the lysozyme-actin complex at a wide range of monovalent salt concentrations (FIG. 3). The structure was obtained using the following methods and materials.

Monomeric G-actin (MW 42,000) was prepared from a lyophilized powder of rabbit skeletal muscle purchased from Cytoskeleton, Inc. (Denver, Col.). The non-polymerizing G-actin solution contained a 5 mM TRIS buffer at pH 8.0, with 0.2 mM CaCl₂, 0.5 mM ATP, and 0.2 mM DTT and 0.01% NaN₃. G-actin (2 mg/ml) was polymerized into F-actin (linear charge density λ_{A}˜−1 e /2.5 Å at pH 7) upon the addition of salt (100 mM KCl). Human plasma gelsolin (Cytoskeleton, Inc., Denver, Col.) was used to control the average F-actin length between 0.1 μm to 10 μm. The filaments were treated with phalloidin (Sigma Aldrich, St. Louis, Mo., MW 789.2) to prevent actin depolymerization and resuspended to a final concentration of ˜26.67 mg/ml using Millipore (Billerica, Mass.) H₂O (18.2MΩ). Hen Egg White Lysozyme (MW 14,300, Sigma Aldrich, St. Louis, Mo.) was dissolved in Millipore H₂O (18.2MΩ) to a final concentration ˜25 mg/ml. Lysozyme carries a pH dependent charge of +9 or +10 and has dimensions of approximately ˜26Å×26Å×45 Å.

The F-actin—lysozyme isoelectric point is found at a concentration ratio of ˜2.5:1 F-actin:lysozyme. X-ray samples were prepared at various F-actin:lysozyme concentration ratios on either side of the isoelectric point, including 2:1, 2.5:1, 3:1, and 5:1. The effect of monovalent salts (KCl & NaCl) on F-actin-lysozyme condensation was investigated by preparing samples at a range of concentrations from 0 mM to 500 mM, so that no matter the ASL ionic concentration, we have the structural solution.

X-ray samples were prepared by sealing F-actin, lysozyme, and monovalent salt solution into 1.5 mm diameter quartz capillaries, followed by mixing and centrifugation. Small angle x-ray scattering (SAXS) experiments were performed at both at Beamline 4-2 of the Stanford Synchrotron Radiation Laboratory (SSRL) as well as at an in-house x-ray source. The incident synchrotron x-rays from the 8-pole Wiggler were monochromatized to 8.98 KeV (λ=1.3806 Å) using a double-bounce Si(111) crystal, focused using a cylindrical mirror. The scattered radiation was collected using a MAR Research charged coupled device (CCD) camera (pixel size=79 μm). For the in-house experiments, incident CuKa radiation (λ=1.54 Å) from a Rigaku rotating-anode generator is monochromatized and focused using Osmic (Auborn Hills, Mich.) confocal multilayer optics, and scattered radiation is collected on a Bruker 2D wire detector (pixel size=105 μm). The 2D SAXS data from both systems are mutually consistent.

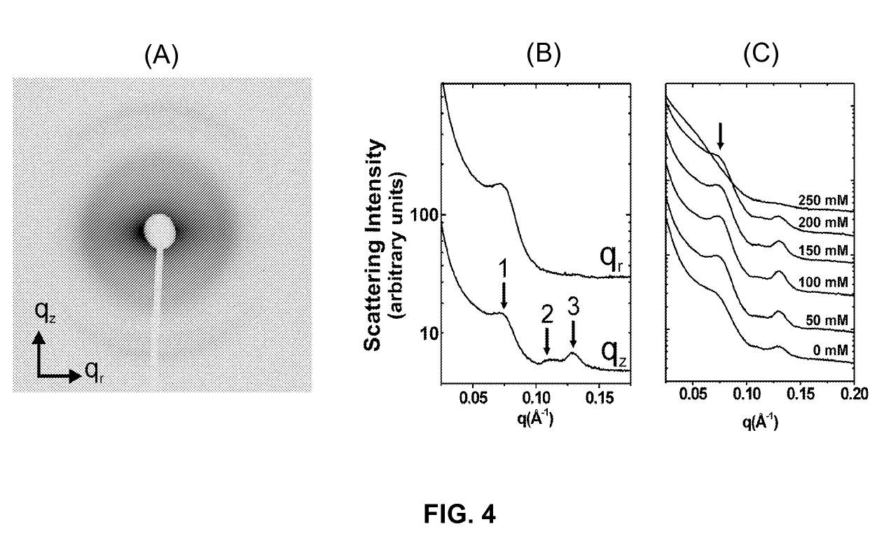

A 2-D diffraction pattern for partially aligned F-actin-lysozyme bundles and its associated 1-D integrated slices along the q_{z} and qᵣ directions are shown in FIGS. 4A & 4B. An examination of the slice along the equatorial (qᵣ) direction shows the actin-lysozyme close pack bundling peak at q=0.07 Å⁻¹. This peak corresponds to an inter-actin spacing of ˜90 Å which proves to be ample space for the presence of lysozyme. This 90 Å inter-actin spacing can be compared to the 70 Å inter-actin spacing for actin condensed with multivalent salts. Angelini et al., 2003. Along the meridional (q_{z}) axis, the actin-lysozyme bundling peak is present (q=0.07 Å⁻¹) along with a weak peak at 0.0113 Å⁻¹ which corresponds to one of the well-characterized actin helix form factors. Most interestingly, however, is the appearance of a peak at ˜0.130 Å⁻¹ in q-space. This peak corresponds to the lysozyme-lysozyme correlation peak which when converted from q-space to real space corresponds to an inter-lysozyme distance of −48.3 Å or roughly the long axis of lysozyme, implying that the lysozyme is close-packed within the F-actin bundles. FIG. 3 shows schematic representations of a condensed F-actin and lysozyme bundle. The inter-actin distance is modeled at 90 Å in these low-resolution density maps. We have observed similar diffraction features in sputum samples collected from CF patients, as described below.

To investigate the effects of monovalent salt on F-actin condensation, a series of high-resolution synchrotron SAXS measurements were performed on F-actin solutions (average length ˜3000 Å). The SAXS data (FIG. 4C) shows an increase in the F-actin-Lysozyme close pack bundling peak with increasing [NaCl] up to ˜150 mM. At concentrations up to ˜150 mM, screening by the monovalent salts results in a strengthened attraction between the oppositely charged macroions. However, at monovalent salt concentrations greater than 150 mM, the trend reverses with the weakening of the close-pack bundling peak. A series of SAXS measurements were also obtained for actin-lysozyme condensation in the presence of increasing concentrations of KCl with similar results. This indicates that the monovalent salt effect is a general effect rather than a cation specific effect. Furthermore, these results are consistent with recent observations in which antibacterial activity is increased as the salt level of the ASL was artificially lowered using a sugar-derivative osmolyte.

Such “in vitro” data can be related to CF mucus in patients by conducting similar experiments on sputum from CF patients. Cystic Fibrosis patients from Carle Clinic in Urbana who have not been treated with DNase voluntarily expectorate approximately 5-10 ml of sputum during respiratory therapy. After collection, the sputum samples are rapidly frozen and stored at −20° C. for later structural analysis experiments.

Obtaining structural information on the supramolecular organization of CF sputum is difficult, since it is highly inhomogeneous, with structures that change over short length-scales (˜10 μm). This problem is solved by using microdiffraction techniques from 3rd generation synchrotron x-ray sources. We performed microdiffraction experiments at beamline 2D-1D-D at the Advanced Photon Source of the Argonne National Laboratory. Monochromatized x-rays at 8.05 keV (λ=1.5498 Å) were focused to a beam size of 0.5 μm×0.5 μm using a Fresnel Au/Si Zone Plate followed by an order sorting aperture of 10 pm diameter. Scattered x-rays were measured using a LN₂ cooled Charge-Coupled Device (CCD) area detector. Samples were mounted on a sample stage with submicron translation control necessary for scanning the samples.

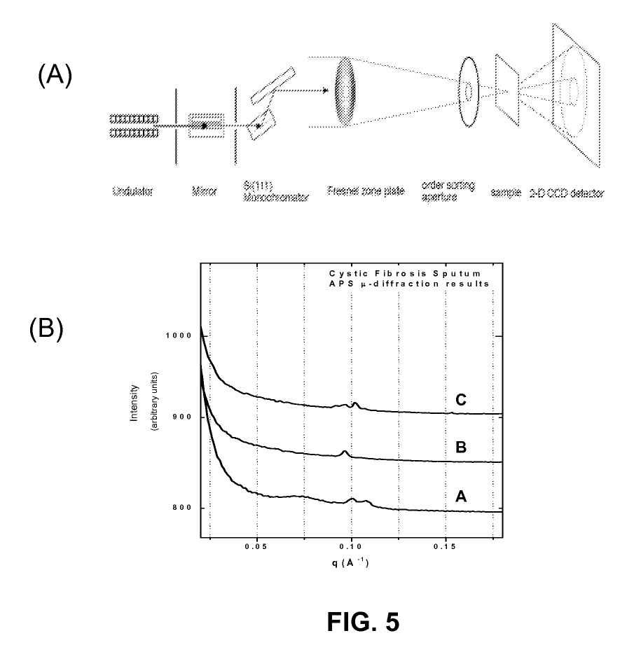

Representative results of the microdiffraction data from CF sputum samples are shown in FIG. 5B. In the 1-D slice labeled A, there is evidence of an F-actin-lysozyme close pack bundling peak at ˜q=0.07 Å⁻¹ as well as the actin helix form factor at ˜q=0.110 Å⁻¹. In slices B & C, however, the actin peaks are not visible, instead we see the appearance of peaks from a condensed phase of unknown origin. The small number of diffraction peaks suggests that a small number of different macromolecular species dominate the bundling process in CF mucus.

The techniques discussed herein identify which antimicrobials are capable of forming bundles with the various anionic polyelectrolytes in the ASL. It is known empirically that in general trivalent ions are required to generate attractions and ‘bundle’ DNA, while only divalent ions are required to ‘bundle’ F-actin and microtubules, while monovalent ions do not ‘bundle’ any of them. We have developed a general biophysical criterion for whether a given cation will induce bundle formation in a wide range of biological polyelectrolytes, based on the cation size and valence relative to the Gouy-Chapman screening length. Butler et al. (2003). These results suggest that below a charge threshold, a cationic ligand can not bind to and condense anionic polyelectrolytes such as DNA and F-actin.

### EXAMPLE 3

### Charge Reduced Antimicrobial Peptides

A continued inflammatory response to chronic and/or repeated infections in the airways leads to the pathological release of cytoskeletal proteins, DNA and other polyelectrolytes in the airways of CF patients. This release of polyelectrolytes cause the electrostatic assembly of large aggregates stabilized by cationic ligands in CF mucus, and results in the sequestration of endogenous antibacterial polypeptides and contributes to the loss of antimicrobial function. In this example, the charge of a native antimicrobial is reduced to minimize sequestration, thereby rescuing antimicrobial efficacy.

The ionic environment of CF mucus is complex. Electrostatics in complex fluids have recently received extensive attention both theoretically and experimentally. Although there is still an unresolved debate on the ionic strength of the ASL (a ˜5 μm thick liquid layer on the surface of the airway epithelium), it is clear that the ASL in CF is enriched in anionic polyelectrolytes. The primary constituents of normal mucus are water, salts, and the glycoprotein mucin. While production of mucus glycoproteins appears to be normal in CF patients, its degree of hydration, which determines the mucus viscosity, is not. This is, at present, not well understood. A number of different mechanisms have been proposed, based on CFTR induced changes in the salt concentrations in the ASL. In addition to the anionic glycoproteins comprising normal mucus, CF mucus contains highly anionic polyelectrolytes such as extracellular filaments produced by colonizing bacteria, as well as F-actin and DNA released from lysed inflammatory cells. The concentration of DNA in CF sputum can be as high as 20 mg/ml, and comprises 4-10% of the dry weight of the sputum. Likewise, F-actin comprises ˜10% of total leukocyte protein, with concentrations reported to be 0.1-5 mg/ml. There is evidence that the altered salt environment in the airways diminishes the activity of native antibacterial proteins. It has also been observed that these anionic polyelectrolytes can bind to and completely inactivate cationic antibacterial proteins.

The increased ionic strength in the ASL of CF patients inhibits the antimicrobial activities of a number of proteins, including lysozyme, β-defensins, and lactoferrin-derived fragments. Bals et al. (1998ab); Smith et al. (1996); Weinberg et al. (1988). For example, although β-defensins are constitutively expressed and induced in CF (Singh et al. (1998)), they do not function properly in the diseased ASL. One common, unifying feature of a wide range of antibiotic peptides is that they are cationic amphiphiles that can interact strongly with the anionic surfaces of microbes, and destabilize their membranes via binding events that are to a large extent electrostatic, and therefore depend on the ionic environment. Therefore, it is important to understand electrostatic interactions in the ASL.

Charge-reduced antibacterial proteins or peptides have a lower binding affinity to anionic polyelectolytes in the ASL and, therefore, lower sequestration levels. Lower sequestration levels correspond to increased overall antibacterial activity in the airway. Although the method of charge-reduction to improve antimicrobial or antibacterial activity in the CF airway is generally applicable to a broad range of antibacterial proteins or peptides including, for example, lysozyme, β-defensins, and lactoferrin-derived fragments, lysozyme is used in the present examples.

Lysozyme is an antibacterial protein found in high concentrations in the CF airway. We have demonstrated how cationic antibacterial proteins such as lysozyme can self-assemble with the anionic polyelectrolytes in the airways such as F-actin into a stable complex and consequently be sequestered. Such sequestration reduces the effectiveness of antibacterial proteins by limiting contact with the target organism, e.g., with the bacterial cell wall. By using synchrotron small angle x-ray scattering (SAXS), we have determined the structure of actin-lysozyme complexes in a wide range of salt concentrations, and now understand the interactions that govern their self-assembly (FIG. 3). Similar structures are observed in sputum samples collected from CF patients, indicating these sequestration complexes exist in vivo (see FIG. 5).

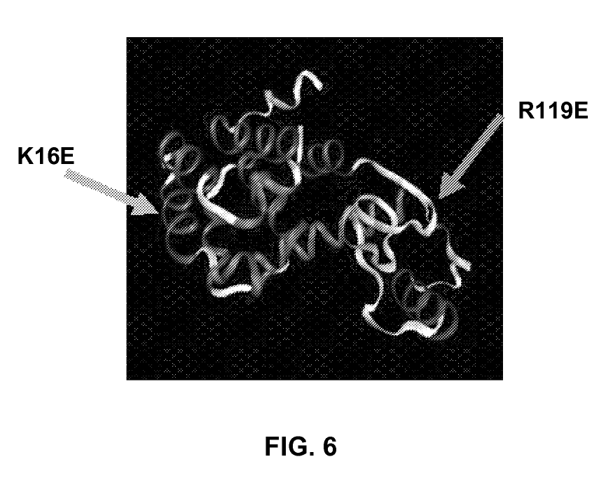

The binding and self-assembly between F-actin and lysozyme can be controlled by modifying the lysozyme charge, as demonstrated by binding studies comparing wild type T4 lysozyme and charge-reduced T4 lysozyme mutants. Wild-type lysozyme has a charge of +9. Using site-directed mutagenesis at two different cationic residues, it is possible to obtain charge +5 mutants at neutral pH. This is accomplished by mutating K16E (lysine to glutamic acid) and R119E (arginine to glutamic acid) (FIG. 6). See Dao-pin et al. (1991). It has been shown that these charge mutants retain most of their antibacterial function.

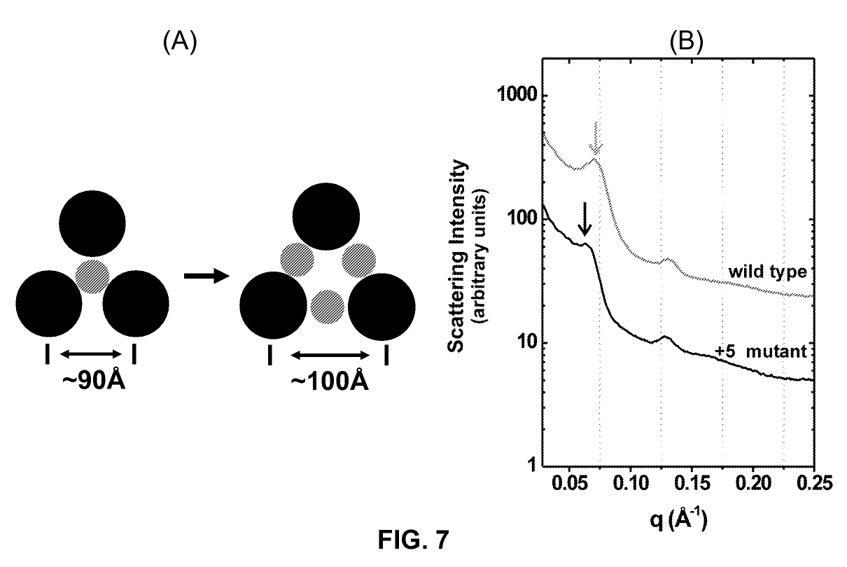

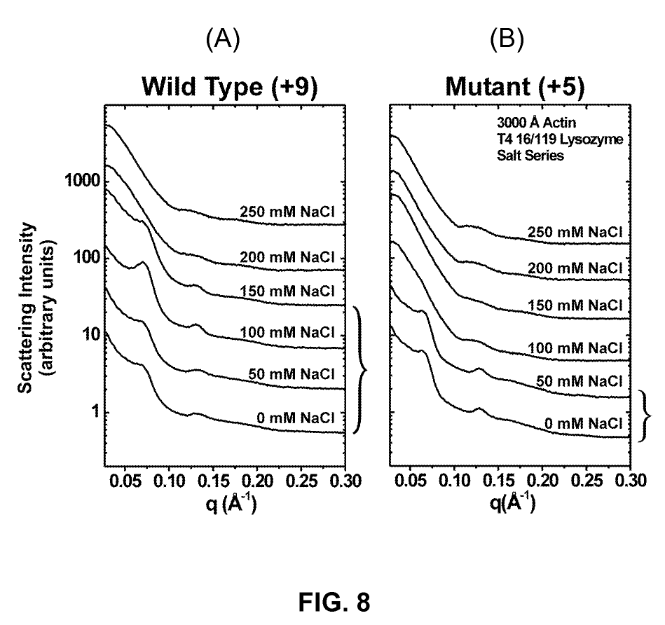

The SAXS data (FIG. 7B) for actin-lysozyme complexes for both the wild-type and mutant lysozymes indicates that the lysozyme has migrated from a 3-fold to a more loosely bound 2-fold bridging site when the charge is reduced (FIG. 7A). This change has been confirmed using molecular dynamics computer simulations. FIG. 8 indicates that this structural change has a profound affect on the relative stability of the complexes at physiological salt conditions.

### Experimental Methodology

Plasmids for bacteriophage T4 wild-type (SEQ ID NO:1) and mutant lysozymes were provided by Professor Brian Matthews at the University of Oregon. The mutant lysozymes included two single mutants, K135E and R154E; four double mutants: K16E/R119E; K16E/K135E; K16E/R154E; K135E/K147E; and one triple mutant: K16E/K135E/K147E. (K=lysine; E=glutamic acid; R=arginine; where, for example, K135E indicates that a lysine residue at position 135 was substituted with glutamic acid). Proteins were expressed and purified according to previously published methodology. Dao-pin, et al. (1991). Purified lysozymes are diluted to working concentrations using ultrapure H₂O (18.2 MΩ; Millipore Corporation, Billerica, Mass.).

Monomeric actin (G-actin) (MW 42 000) was prepared from a lyophilized powder of rabbit skeletal muscle. (Cytoskeleton, Inc., Denver, Col.). The non-polymerizing G-actin solution contained a 5 mM Tris buffer at pH 8.0, with 0.2 mM CaCl₂, 0.5 mM ATP, 0.2 mM DTT, and 0.01% NaN₃. G-actin (2 mg/ml) was polymerized into F-actin (linear charge density λ_{A}≈1e/2.5 Å at pH 7.0) by the addition of monovalent salt (100 mM Nacl final concentration). Human plasma gelsolin, an actin severing and capping protein (Janmey et al., 1986) (Cytoskeleton, Inc.), was added at a gelsolin:actin monomer molar ratio of 1:370 to restrict the length of the F-actin polymers to approximately 1 μm. The filaments were treated with phalloidin (MW 789.2; Sigma Aldrich, St. Louis, Mo.) to prevent actin depolymerization. F-actin gels were ultracentrifuged at 100 000 g for 1 h to pellet the filaments. After the removal of the supernatant buffer solution, the F-actin was resuspended in ultrapure H₂O (18.2 MΩ; Millipore Corporation).

Stock solutions of NaCl and KCl were prepared by mixing in ultrapure H₂O. In order to ensure uniform mixing and to decrease error associated with pipetting small volumes, lysozyme was pre-mixed with monovalent salt solutions in 100 μl aliquots. The isoelectric actin-lysozyme complexes were prepared by flame sealing F-actin with lysozyme-monovalent salt solutions in 1.5 mm quartz capillaries (Hilgenberg GmbH; Malsfeld, Germany) and mixing thoroughly by centrifugation. The approximate sample volume in the capillary was 30 μl. The final F-actin concentration was ˜4.3 mg/ml while the final wild-type lysozyme concentration was ˜4.2 mg/ml and double mutant concentration was ˜2.3 mg/ml. A series of samples are prepared with the final monovalent salt concentration ranging from 0 mM to 500 mM.

SAXS measurements were performed using the in-house x-ray source located in the Frederick Seitz Materials Research Laboratory (Urbana, Ill.), beamline 4-2 at the Stanford Synchrotron Radiation Laboratory (SSRL; Palo Alto, Calif.), and BESSERC beamline 12-ID-C at the Advanced Photon Source (APS; Argonne National Laboratory, Argonne, Ill.). For the in-house experiments, incident Cu Ka radiation (λ=1.54 Å) from a Rigaku (The Woodlands, Tex.) rotating-anode generator was monochromatized and focused using Osmic (Auborn Hills, Mich.) confocal multilayer optics, and scattered radiation was collected on a Bruker AXS (Madison, Wis.) 2D wire detector (pixel size=105×105 μm²). For the SSRL experiments, incident synchrotron x-rays from the eight-pole wiggler were monochromatized to 8.98 keV using a double-bounce Si(111) crystal (λ=1.3806 Å) and focused using a cylindrical mirror. The scattered radiation was collected using a MAR Research (Evanston, Ill.) charge-coupled device camera (pixel size=79×79 μm²). For the APS experiments, incident x-ray wavelength was set at λ=1.033 Å by a double-crystal Si(111) monochromator and focused using a flat-focusing monochromatic mirror. The scattered x-rays were collected using a two-dimensional mosaic CCD detector (pixel size=79×79 μm²; MAR Research). The sample-to-detector distances were set such that the detecting range is 0.03<q<0.25 Å⁻¹, where q=(4πsinθ/λ, λ is the wavelength of the incident beam, and 2θ is the scattering angle. The 2D SAXS data from all set-ups have been checked for mutual consistency.

Typically, the precipitated F-actin—lysozyme complex is compacted into a dense pellet during mixing. These pellets consist of many coexisting domains of actin bundles locally oriented along different random directions, as indicated by the “powder averaging” of the diffraction pattern and the associated loss of orientational information. In order to minimize these effects, a small (300×300 μm²) x-ray beam is used to obtain diffraction information on locally aligned domains within the pellet.

### EXAMPLE 4

### Effect of Salt Concentration on Antimicrobial Sequestration

FIG. 8 shows the SAXS spectra for electrostatic complexes formed between F-actin and the two types of T4 lysozyme, the wild-type (FIG. 8A) and charge-reduced (FIG. 8B) lysozyme. The self-assembly of actin-lysozyme bundles is maximized between 100 mM and 150 mM monovalent salt (only NaCl shown) for wild-type lysozyme. For the mutant (+5 charge), however, the maximum is decreased to about between 0 mM and 50 mM or less. At the range of salt concentrations expected in the CF airway (50 mM-150 mM), the lysozyme unbinds from the actin, and is no longer sequestered. Experiments indicate a similar unbinding effect with DNA, which is also found in the airway. Liberating lysozyme from bound complexes in this manner can at least partially restore lysozyme antimicrobial activity in the airway. This basic strategy is amenable to other antimicrobials including, for example, lactoferrin, β-defensins, and fragments thereof.

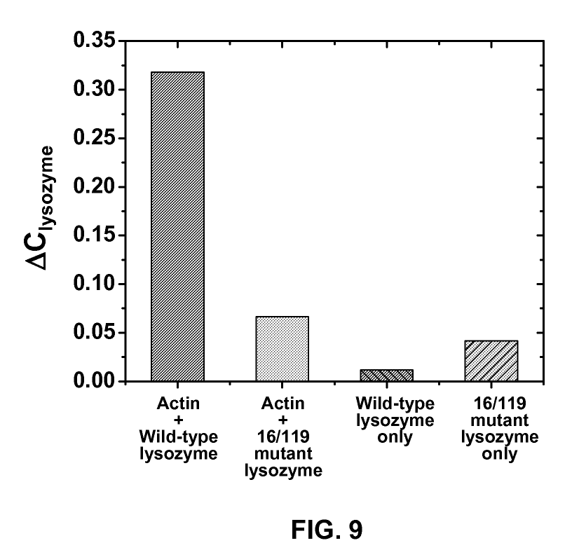

To further explore the extent of lysozyme sequestration by actin, equilibrium dialysis experiments (FIG. 9) were performed using microdialyzers (Harvard Apparatus; Boston, Mass.) containing two 100 μl chambers separated by a 50,000 dalton molecular mass cutoff cellulose acetate membrane. The “sample” chamber contained protein solutions suspended in 100 mM NaCl/2 mM Tris buffer; the “assay” chamber contained only 100 mM NaCl/2 mM Tris buffer. The protein solutions were either actin-lysozyme complexes or controls with only lysozyme. Protein solutions were allowed to dialyze against the NaCl/Tris buffer for 6 days to ensure complete dialysis. Protein concentrations were measured using UV-VIS spectroscopy at 280 nm. For the actin-lysozyme complexes, only the “assay” concentration was measured due to the presence of actin in the “sample” chamber. As seen in FIG. 9, the deviation from equilibrium for wild-type lysozyme sequestered by actin is greater than for mutant lysozyme complexed with lysozyme, which supports the x-ray evidence indicating that mutant lysozyme is released from actin complexes at physiological salt concentrations.

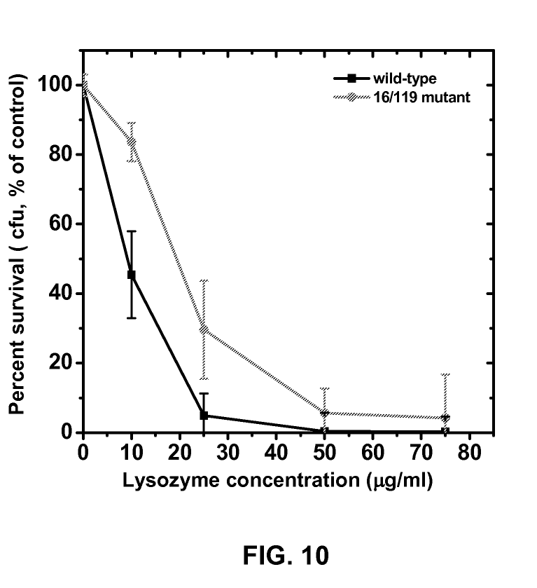

The release of charge-reduced antimicrobials was further verified by antibacterial activity assays similar to that shown in FIG. 10. Pseudomonas aeruginosa and Staphylococcus aureus, the two most common colonizers of CF lungs, as well as coagulase-negative Staphyloccus, an organism found in normal nasal mucosa, is grown and resuspended in appropriate medium (e.g., Luria-Bertani (LB) medium or Mueller-Hinton (MH)) and incubated with and without extracts from dissolved complexes from both in vitro samples, and from CF sputum. In FIG. 10, results from bactericidal susceptibility assays on Pseudomonas aeruginosa (PAO1) are shown. PAO1 were grown from an overnight culture in cation-adjusted Mueller-Hinton (MH) broth to mid-log phase and harvested by desktop centrifugation. The bacteria were resuspended in phosphate buffered saline (PBS, 10 mM Na₂HPO₄/100 mM NaCl, pH=7.4) and diluted so that ˜10⁵ colony forming units (cfu) are present in the final volume of 150 μl of the assay buffer (PBS). Bacteria were incubated with lysozyme in sterile, 96-well flat bottom polypropylene dishes while shaking for 3 hours at 37° C. Following the incubation period, bacteria were serially diluted, dropped on MH agar plates, and incubated ˜18 h at 37° C. Cfu's were determined by using standard plate-counting methods. As seen in FIG. 10, the double mutant lysozyme shows similar efficacy as the wild-type lysozyme with increasing lysozyme concentrations. This indicates that the site-directed mutagenesis does not significantly affect the antimicrobial efficacy of the charge-modified mutant lysozyme as substantial activity is retained.

The antimicrobial activity of wild-type lysozyme decreases with increasing salt (Travis, et al., 1999); this decrease can be compensated with larger lysozyme concentrations. The influence of salt on the activity of the charge-reduced mutants can be assessed as disclosed herein, and compared to that of the wild-type. Data (not shown) indicate that while the antimicrobial activity of wild type lysozyme is higher than that of the charge-reduced mutant at low salt levels (˜10 mM NaCl), the difference in activity between the two decreases dramatically as the salt level is increased. The activity of the two types of lysozyme is comparable at physiological salt levels, so that the mutant lysozyme will show a significant increase in antimicrobial activity due to the lower level of mutant sequestration in the airway relative to the level of wild-type sequestration. Those of ordinary skill in the art will recognize that this approach can be utilized to manufacture human-recombinant versions of such mutants for delivery to the airways, e.g., in aerosolized form, to counter long-term infections.

### EXAMPLE 5

### Entropy-Optimized Surfactants for Decreasing Aminoglycoside Antibiotic Sequestration.

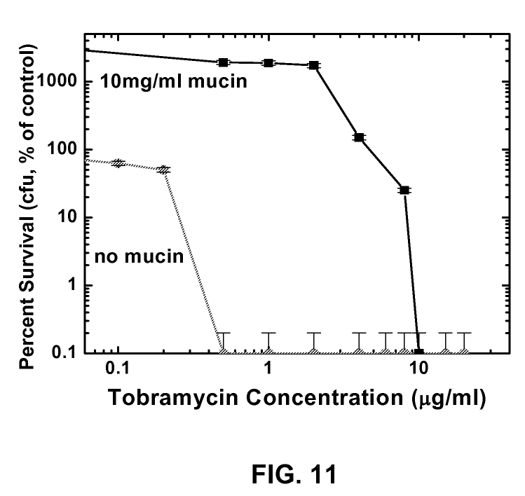

The aminoglycosides are a family of potent antibiotics that are made of highly cationic sugars, and are used for a variety of biomedical conditions, such as cystic fibrosis. Due to their high positive charge, however, they often become sequestered via electrostatic interactions with oppositely charged molecules preventing them from reaching their intended target, thus decreasing their efficiency. This is illustrated in FIG. 11, which illustrates the efficacy of an aminoglycoside antibiotic (tobramycin) is decreased in the presence of mucin.

We have developed a general strategy to use rationally-designed positively-charged surfactants (similar to those which are currently being used in for gene-therapy), to coat the negatively charged polymers, thus freeing the aminoglycoside antibiotics and allowing them to fulfill their antibacterial function. This is accomplished by designing surfactant formulations that have the appropriate amount of charge and curvature, so that they can maximize counterion entropy gain upon binding, and thereby optimally coat anionic polymers in the airway, thus preventing antibiotic binding. These surfactants can be used to also inhibit binding of the aminoglycoside antibiotics to the glycoproteins present in the airway. This method can also be applied to other highly infected systems as well as for other antimicrobials and antibiotics.

One of the most common bacterial infections in CF patients is that of Pseudomonas aeruginosa which is primarily treated by administration of aminoglycoside antibiotics, specifically tobramycin. It has been observed, however, that the anionic polyelectrolytes present in CF sputum can bind to and completely inactivate these cationic antibiotics (see FIG. 11). The biological accessibility of the tobramycin can be as low as 1/20th of the available dose due to this sequestration.

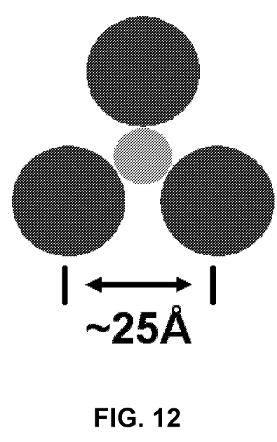

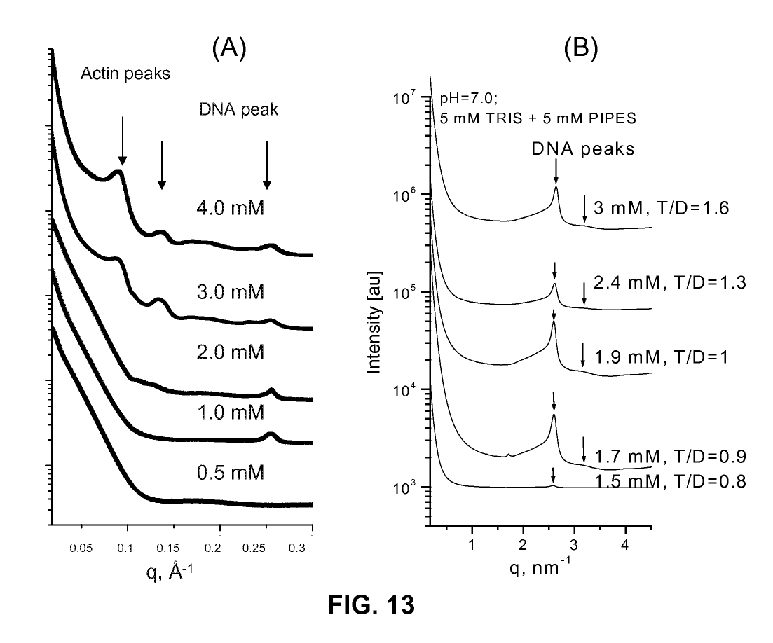

Tobramycin is a multivalent cationic anti-pseudomonal antibiotics (charge of +5) currently used to treat bacterial infections in the airways of CF patients. As discussed above, multivalent cations can self-assemble with the anionic polyelectrolytes in the airways (e.g. F-actin and DNA) to form a stable complex (FIG. 1). Zribi et al. (2005). Such self-assembly results in sequestration of cationic antibiotics, and an associated decrease in antibiotic efficacy. Zibri et al. show that the multivalent ion, spermidine preferentially associates with the more highly charged DNA over F-actin. Similarly, tobramycin preferentially interacts with DNA over F-actin (FIG. 13). Thus, we first target prevention of sequestration of tobramycin by DNA. By using synchrotron small angle x-ray scattering (SAXS), we have determined the structure of DNA-tobramycin complexes (FIG. 12). This complex is typical of DNA condensed by multivalent counterions. FIG. 13 shows SAXS data for various tobramycin charge to DNA charge ratios (0.81<T/D<1.6)

We demonstrate that the binding and self-assembly between DNA and a charged antimicrobial such as tobramycin can be limited by adding a solution of cationic lipids to the DNA-tobramycin suspension. Cationic lipids are surfactants that are known to form complexes with many different types of anionic polymers including DNA and F-actin. Cationic lipid vesicles are used in other applications, e.g., as gene carriers in clinical trials of non-viral gene therapy. Ewert et al. (2004). In one embodiment, a mixture of two lipids is used: DOPE, which is one of the main neutral lipids in use in gene therapy applications, and DOTAP, which has a positively charged hydrophilic head. A mixture of the two lipids creates an appropriate ratio of charge and lipid curvature in order to wrap the lipid membranes around the DNA. It has been shown that by mixing these lipids together in a ratio from 100:0 to 35:65 of DOTAP:DOPE, the lipids form a lamellar complex with DNA. Raedler et al. (1997). By mixing the lipids together in a ratio from 35:65 to 10:90 of DOTAP:DOPE, the lipids form an inverted-hexagonal phase with the DNA because of the curvature of the DOPE. Koltover et al. (1998). For our experiments we chose ratios within the range of each of the two phases, 70:30 DOTAP:DOPE and 25:75 DOTAP:DOPE, to identify the behavior of the lamellar and inverted hexagonal phases.

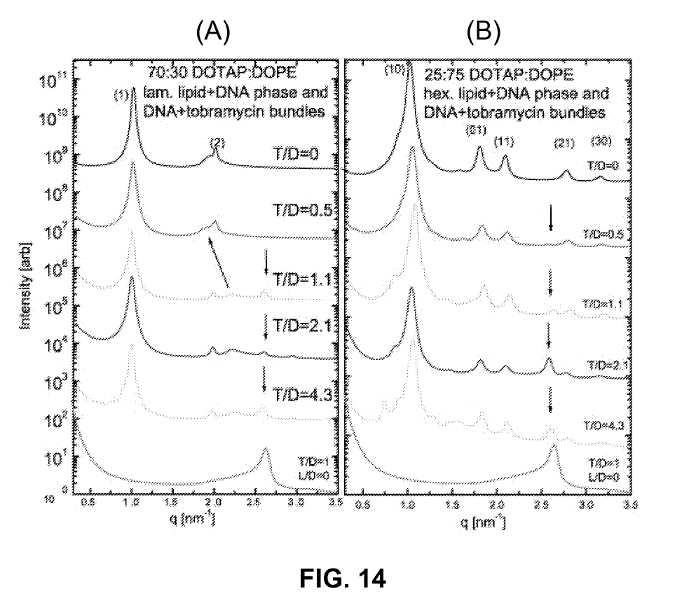

A series of SAXS experiments were conducted at physiological salt and pH conditions in which the cationic lipid mixtures were added to DNA+tobramycin suspensions. FIG. 14 shows the SAXS spectra for electrostatic complexes formed between DNA, tobramycin and two different lipid mixtures. At low tobramycin concentrations, the DNA-tobramycin bundling peak disappears (arrows). This indicates that tobramycin can be unsequestered by the addition of cationic-lipids to CF airways. Both the 70:30 and the 25:75 lipid mixtures form complexes with DNA even in the presence of tobramycin. Additionally, because the 25:75 lipid mix forms an inverted hexagonal complex with the DNA leaving no room for tobramycin in the lipid-DNA complex, this structure may release even more tobramycin than the lamellar structure. In both experiments the lipid concentration is set to be sufficient to neutralize the DNA charge (L/D=1). The DNA interacts almost entirely with the lipids and not the tobramycin up until a tobramycin concentration that is high enough to neutralize the DNA charge (T/D=1). In this range, the tobramycin concentration is too low to form the same DNA-tobramycin complexes shown in FIG. 12, but is indeed equivalent to the tobramycin concentrations found in CF sputum which range from 10-100 μg/ml, corresponding to T/D=0.01-0.13 in our experiment. In this concentration range, DNA in sputum plays a role in sequestering tobramycin. Mendelman et al. (1985); Hunt et al. (1995); Rampal et al. (1988). Therefore, we find that for a lipid concentration greater than or equal to that required for DNA charge neutralization, the DNA is coated by the lipids, and the tobramycin is not sequestered. Furthermore, we find that when the ratio of cationic lipid charges to tobramycin charge is greater than one, the tobramycin can be unsequestered, even if the tobramycin concentration is itself high enough to form the complexes illustrated in FIG. 12 (T/D>1).

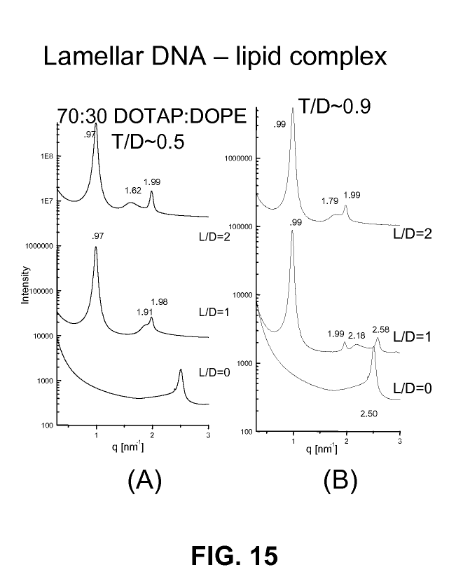

This basic strategy of liberating tobramycin bound to DNA complexes by adding lipid-based surfactants can function at many different DNA and tobramycin concentration ranges to restore partial tobramycin antimicrobial activity in the airways. For example, FIG. 15 shows SAXS data for two different tobramycin to DNA charge ratios (T/D=0.5 in (A) and 0.9 in (B)) and for a variety of lipid concentrations (L/D=0, 1, and 2). FIG. 15 illustrates that increasing lipid concentration decreases sequestration of tobramycin. Because the lipid concentration needed for this strategy depends on the DNA concentration present, this technique is particularly effective for early-stage bacterial infections, where the DNA concentration in the sputum is still low. Since adjusting the composition of the lipids to change curvature and charge is facile, this basic strategy is applicable for releasing tobramycin from the other anionic polymers, including for example, actin and mucin. By varying the lipids or surfactant mixtures, this effect is general and can be tailored to a variety of antibiotics sequestered by DNA, actin, mucin and/or other anionic polymers.

This method can adequately function even if not all the DNA or actin or mucin in the airways is bound. In one embodiment, these surfactant formulations are administered as an aerosol to pacify the charged surfaces immediately before the administration of antibiotics. Such a method increases the efficacy of the administered antibiotic compared to when an antibiotic is administered without these surfactant formulations.

In vitro bactericidal assays can be used to quantify the accessibility of the tobramycin to work as an antibacterial in the presence of the lipid+DNA complexes (FIG. 11). In addition, the effects of utilizing lipid-based surfactant on other anionic polymers (e.g. F-actin and mucin) in tobramycin sequestration can be examined using the methodology disclosed herein. Optimizing lipid curvature and charge results in maximal unbinding of tobramycin sequestered by DNA. The experiments disclosed herein used a mixture of DOTAP and DOPE lipids. However, those skilled in the art recognize that the principles illustrated by the DOTAP and DOPE lipids are general, so that the methods disclosed herein are applicable for other surfactants and lipids (including commercially available ones) so as to optimize the surfactant mixture.

### Experimental Methodology

For our studies of the sequestration of aminoglycoside antibiotics by model CF sputum and subsequent release by the administration of surfactants we used the following materials. The aminoglycoside antibiotic used in our experiments was Tobramycin (Sigma Aldrich), commonly used to treat infections of Pseudomonas aeruginosa in CF patients. Tobramycin has been shown to be sequestered by DNA, F-Actin, and mucin present in CF mucous. For the DNA+tobramycin experiments we used Calf thymus (CT) DNA (USB Corp. Cleveland Ohio), purified it using standard techniques, and resuspended it in an aqueous solution of 100 mM NaCl 5 mM Tris and 5 mM PIPES adjusted to pH 7. DNA has a charge of approximately −2/base pair.

For the F-actin+tobramycin experiments, lyophilized rabbit skeletal muscle monomeric G-actin (Cytoskeleton, Inc., Denver, Col.), was resuspended in non-polymerizing G-actin solution containing 5 mM Tris buffer at pH 8.0, with 0.2 mM CaCl₂, 0.5 mM ATP, 0.2 mM DTT, and 0.01% NaN₃. G-actin (2 mg/ml) was polymerized into F-actin (linear charge density λ_{A}=1 e/2.5 Å at pH 7.0) by the addition of monovalent salt (100 mM KCl final concentration). Human plasma gelsolin, an actin severing and capping protein (Janmey et al. (1986) (Cytoskeleton, Inc.), was added at a gelsolin:actin monomer molar ratio of 1:370 to restrict the length of the F-actin polymers to approximately 1 μm. The filaments were treated with phalloidin (MW 789.2; Sigma Aldrich, St. Louis, Mo.) to prevent actin depolymerization. F-actin gels are ultracentrifuged at 100 000 g for 1 h to pellet the filaments. After the removal of the supernatant buffer solution, the F-actin was resuspended in ultrapure H₂O (18.2 MΩ; Millipore Corporation). For the mucin+tobramycin experiments we used porcine stomach mucin (Sigma Aldrich). Similar studies can be conducted using human respiratory mucous. Porcine mucin was resuspended in aqueous solution and autoclaved for 5 minutes to ensure sterility. Mucin is found in respiratory mucous at concentrations of 1-5 mg/ml and is highly negatively charged, though the exact charge is unknown due to the polydispersity of the structure of the polymer.

Surfactant materials used in the present experiments were a mixture of two lipids DOTAP (1,2-Dioleoyl-3-Trimethylammonium-Propane) and DOPE (1,2-Dioleoyl-sn-Glycero-3-Phosphoethanolamine) (Avanti Polar lipids, Alabaster Al.) in mass ratios of about 70:30 and about 25:75. DOTAP is a cationic lipid (charge of +1) with no curvature, and DOPE is a neutral lipid with negative curvature. Briefly, the lipids were first dissolved in chloroform, then dried under nitrogen and redissolved in an aqueous solution. The aqueous salt solution was then sonicated to form unilamellar lipid vescicles, and filtered through 0.2 micron pores.

Aminoglycoside antibiotic sequestration by negatively charged bio-polymers in respiratory mucous of CF patients was measured via two techniques. The first method was to use x-ray diffraction techniques to discern the structure of suspensions of mixtures of biopolymers and antibiotic. Specific x-ray diffraction studies were done to determine the structure of mixtures of the DNA with tobramycin and the lipid mixture. The isoelectric DNA-lipid-monovalent salt solutions were sealed in 1.5 mm quartz capillaries (Hilgenberg GmbH; Malsfeld, Germany) and mixed thoroughly by centrifugation. The approximate sample volume in the capillary was 30 μl-60 μl. SAXS experiments were performed as described previously. Tobramycin has charge of approximately +5 at physiological pH, whereas DNA is negatively charged, thus when the ratio of net DNA charge is approximately equal to the net tobramycin charge (T/D˜1), the tobramycin condenses the DNA. The resulting structure is a bundle of DNA. The structures observed via x-ray diffraction indicate whether or not the tobramycin is sequestered with the DNA or free in suspension. The present invention uses positively charged lipids to replace the tobramycin in these bundles, thereby increasing the efficacy of the antibiotic. Consequently, our experiments used a fixed DNA concentration of 3 mg/ml (x-ray experiments) and a varied concentration of tobramycin and/or lipid. Concentrations are varied such that the ratio of positive to negative charges is known. Similar x-ray diffraction studies can be conducted wherein mucin, instead of DNA, is the sequestration agent to examine mucin+tobramycin+lipid complexes and kinetics.

Tobramycin sequestration was also measured via bacteriological killing assays. In these bacterial killing assay experiments Pseudomonas aeruginosa (PA01), the primary bacteria responsible for infection in CF patients, was incubated in the presence of mucin, DNA, tobramycin, lipid or various mixtures of these four materials. Killing assays were performed in the following manner: PA01 was grown in cation adjusted Mueller-Hinton media to a concentration greater than 5×10⁸, sedimented by centrifugation and resuspended in a buffered solution of 5 mM Tris 5 mM PIPES and 100 mM NaCl at pH 7.2. A quantity of ×10⁵ c.f.u. of PAO1 were incubated for 3.5 hours in the buffered salt solution with varied concentrations of tobramycin, DNA, mucin, and lipids. The mucin concentrations were either 0 mg/ml (control) or 10 mg/ml which is near the physiologically measured concentrations of mucin. Lipids added to the bacterial solution were added in varying concentrations in order to determine the amount needed to release tobramycin from its strong interaction with mucin and/or DNA. The bacterial solution was serially diluted and plated on MH-agar plates. Plates were incubated overnight and the number of bacterial colonies counted. The minimal inhibitory concentration (MIC) of tobramycin for these assays is approximately 5 μg/ml.

### EXAMPLE 6

### Lipid Formulations for Decreasing Antimicrobial Sequestration.

Based on the disclosure in this application, we demonstrate the applicability of using lipids of any size chain length to interact with substances that can act as sequestering agents such as DNA and F-actin. Thus in addition to DOTAP:DOPE lipid compositions, other lipids of are adaptable for use in embodiments of the invention. For example, PC, PE and TAP lipids of any chain length can be employed to prevent antimicrobial sequestration.

When DNA is mixed with similar cationic lipid mixtures of DOTAP and DOPC, 1,2-Dioleoyl-sn-Glycero-3-Phosphocholine, and when the DOTAP:DOPC ratio varies from 100:0 to 10:90, a lamellar lipid-DNA complex identical to that exhibited by DOTAP:DOPE mixtures at a ratio of 70:30 is measured. Correspondingly, a positively-charged antimicrobial such as tobramycin can be excluded from this DOTAP: DOPC phase in the same way that it is excluded from the DOTAP:DOPE lamellar phase due to the similarity of structural attributes.

SAXS experiments have also shown that cationic lipid mixtures of DLTAP:DLPC, DOTAP:DOPC, and DNTAP:DNPC (dilauryl trimethyl ammonium propane: dilauryl trimethyl phosphatidylcholine, dioleoyl trimethyl ammonium propane: dioleoyl trimethyl phosphatidylcholine, and dinervonyl trimethyl ammonium propane: dinervonyl trimethyl phosphatidylcholine, respectively) for ratios from 100:0 to 10:90 will all form complexes with F-actin. These complexes show that the F-actin is coated by lipids (FIG. 2B), shielding it from interaction with cationic antimicrobials such as tobramycin and lysozyme. In the presence of these lipid solutions, monovalent ions, like Na⁺, and K⁺, which are typically present in the ASL in 100 mM quantities, can condense the actin rods alone within the lipid layers, leaving no room for the antimicrobials to be sequestered. In these F-actin/lipid complexes the lipid to actin charge ratio required is only L/A=0.23; therefore, only a small amount of lipid may be required to begin to coat the F-actin.

Other specific cationic surfactant compositions which display the same interaction with DNA, and can decrease sequestration of antimicrobials, include the following: Didodecyldimethylammonium bromide (DDAB); Cetyltrimethylammonium bromide (CTAB); Cetyltrimethylammonium bromide (CTAB): 1-palmitoyl-2-hydroxy-sn-glycero-3-phosphocholine (PHGPC) from 20:80 to 80:20; and DLTAP:DLPC, DOTAP:DOPC, and DNTAP:DNPC (dilauryl trimethyl ammonium propane: dilauryl trimethyl phosphatidylcholine, dioleoyl trimethyl ammonium propane: dioleoyl trimethyl phosphatidylcholine, and dinervonyl trimethyl ammonium propane: dinervonyl trimethyl phosphatidylcholine, respectively) from 100:0 to 10:90.

Further lipid compositions that can be utilized are listed in Table 1. These lipids have the same hydrophilic head structure but differ in hydrocarbon chain length.

TABLE 1
Lipid compositions.

| Type of | |
|---|---|
| analogs | Description |
| DOPE | 1,2-Diarachidonoyl-sn-Glycero-3-Phosphoethanolamine |
| | 1,2-Didocosahexaenoyl-sn-Glycero-3-Phosphoethanolamine |
| | 1,2-Dielaidoyl-sn-Glycero-3-Phosphoethanolamine |
| | 1,2-Dihexanoyl-sn-Glycero-3-Phosphoethanolamine |
| | 1,2-Dioctanoyl-sn-Glycero-3-Phosphoethanolamine |
| | 1,2-Dihexanoyl-sn-Glycero-3-Phosphoethanolamine |
| | 1,2-Dilauroyl-sn-Glycero-3-Phosphoethanolamine |
| | 1,2-Dimyristoyl-sn-Glycero-3-Phosphoethanolamine |
| | 1,2-Dipalmitoyl-sn-Glycero-3-Phosphoethanolamine |
| | 1,2-Dipalmitoleoyl-sn-Glycero-3-Phosphoethanolamine |
| | 1,2-Diheptadecanoyl-sn-Glycero-3-Phosphoethanolamine |
| | 1,2-Dicapryl-sn-Glycero-3-Phosphoethanolamine |
| DOTAP | 1,2-Dimyristoyl-3-Trimethylammonium-Propane |
| | 1,2-Dipalmitoyl-3-Trimethylammonium-Propane |
| DOPC | 1,2-Dimyristoleoyl-sn-Glycero-3-Phosphocholine |
| | 1,2-Dimyristelaidoyl-sn-Glycero-3-Phosphocholine |
| | 1,2-Dipalmitoleoyl-sn-Glycero-3-Phosphocholine |
| | 1,2-Dipalmitelaidoyl-sn-Glycero-3-Phosphocholine |
| | 1,2-Dieicosenoyl-sn-Glycero-3-Phosphocholine |
| | 1,2-Dierucoyl-sn-Glycero-3-Phosphocholine |
| | 1,2-Dinervonoyl-sn-Glycero-3-Phosphocholine |
| | 1,2-Dipetroselinoyl-sn-Glycero-3-Phosphocholine |
| | 1,2-Dielaidoyl-sn-Glycero-3-Phosphocholine |

### EXAMPLE 7

### Charge-Modified Human Lysozymes.

The human body produces a variety of peptides and proteins, such as lysozyme and lactoferrin, to fight infection such as bacterial infection in the lung. While these molecules are very effective antibacterial agents, they carry high number of positive charges and tend to be sequestrated by negatively charged polyelectrolytes naturally produced from the inflammatory response, such as actin and DNA from lysed cells. A desirable “non-stick” version of an antibacterial peptide/protein will retain its native level of activity but would have reduced electrostatic interaction with the polyelectrolytes. We have developed approaches for making charge-engineered human lysozymes. Two different protein engineering approaches have been used. The first is to engineer reduced-charged mutants of human lysozyme; this is analogous to our efforts described herein regarding T4 lysozyme. The second approach is to reduce charge via a tag. A tag is connected to the lysozyme, e.g. by direct conjugation or by employing an optional spacer. A spacer can be used to prevent the charge-balancing tag from folding back to further associate with the protein itself. Preferably the tag is introduced sufficiently far from the enzyme active site so that the protein activity is not affected. This second approach has an advantage of allowing for facile scale-up in production due to a simpler protocol for purification.

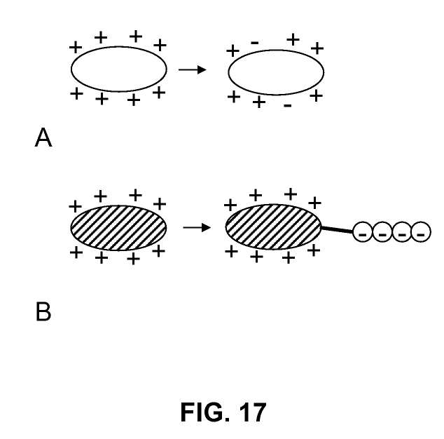

As depicted in FIG. 17, these protein engineering approaches can be used to reduce the net positive charges on a given protein. In the charge reversal approach, positively charged amino acids can be mutated into negatively charged amino acids, thus each mutation can reduce 2+ charges. A possible drawback of this approach is that such mutation can potentially disrupt the protein structure and alter the activity of the protein. In the charge balancing approach, negatively charged amino acids are tagged to the protein, such as to either the N- or C-terminus. Since this approach does not alter the protein structure a priori, it is less likely to affect the activity of the protein.

Human lysozyme was selected as a suitable candidate for developing novel compositions and methods. The native molecule is a small protein with 130 amino acids and a net charge of +9. Charge reversal mutants of T4 lysozymes as described herein can lead to reduced bacterial killing activity for gram-negative bacteria such as E. Coli and Pseudomonas aeruginosa, even though minimal reduction or even enhancement of killing activity has been observed for gram-positive bacteria for certain mutants. We have investigated the alternate strategy of a ‘charge balancing’ approach with the goal of reducing the net positive charge on human lysozyme while preserving antimicrobial activity. The expression charge-balancing does not necessarily imply an equality of balance in overall net charge such as in a state of neutrality.

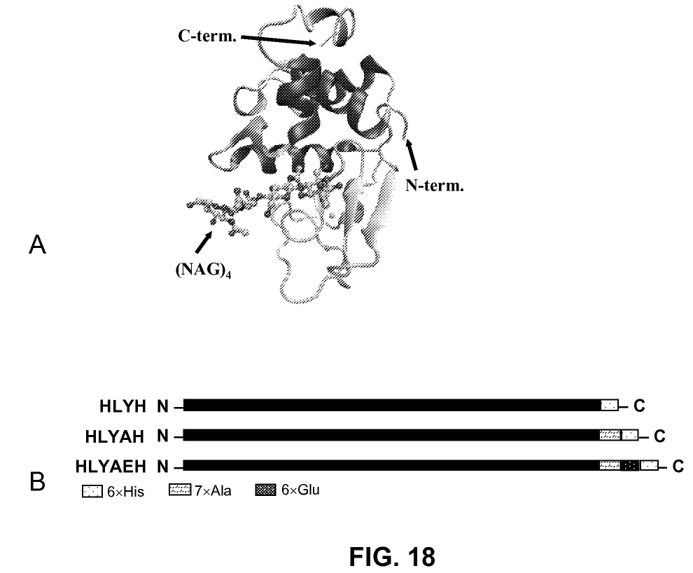

Several aspects of engineering charge balancing lysozyme mutants have been incorporated into this strategy. One aspect is the selection of a preferred location for attaching the negatively charged substance(s). The core of lysozyme's bacteria killing activity lies in its active site, which forms a cleft that hydrolyzes the bond between N-acetyl muramic acid (NAM) and N-acetylglucosamine (NAG) in bacteria cell walls, or the bond between NAG and NAG in fungi cell walls. As shown in FIG. 18, the active site cleft extends towards the N-terminus of the protein, whereas the C-terminus is far away from it (Song et. al, J. Mol. Biol. 244:522-540, 1994). Therefore the C-terminus has been selected as the attaching point for the charge balancing derivatives.

A second consideration for the charge balancing mutant engineering is a spacer between the attached charge-balancing moiety/tag and the lysozyme. A particular factor is how close two charges must be on a given macroion before it generates electrostatic attractions between polyelectrolytes (Butler et al., Phys. Rev. Lett. 2003). We therefore designed a tag of an optimized length with an optimized pattern of charges for attachment to a given antimicrobial so that it will disrupt packing into polyelectrolyte complexes while retaining its full bacteria killing activity.

For a particular analog, a third aspect is the possible necessity to prevent the negatively charged amino acid tag from folding back onto the protein itself. Under such requirements a rigid spacer is desirable, and we have designed a spacer sequence of seven alanine residues. Among the 20 amino acids, alanine has the highest propensity to form alpha-helical structure, which is highly rigid. An (Ala)₇ sequence is expected to form about two turns of alpha-helix, making it a rigid spacer with a length of about 10 Å. The other advantage of an alanine spacer is that it is neither hydrophobic nor hydrophilic. As a result, this spacer sequence is less likely to interact with the lysozyme or the tag. Other tags are developed based on the potential for alanine-rich sequences to subject the labeled proteins to intracellular degradation in bacteria; this may influence the choice of expression system. A further consideration is protein purification, which can facilitate production scale-up. We add a tandem of six histidines (6×His) to the C- or N-terminus of the new mutant lysozymes, and then use a Ni²⁺loaded matrix to purify the histidine-tagged protein based on this affinity tag. At pH 7-7.5, this 6×His tag is neutral and unlikely to impart additional positive charges on the constructed proteins. Based on these considerations, we have constructed three charge-balanced human lysozyme variants, designated HLYH, HLYAH, and HLYAEH (HLYH: A six-histidine (6×His) tag is attached to the C-terminal end of human lysozyme; HLYAH: a spacer sequence of seven alanines (7×Ala) is attached to the C-terminal end of human lysozyme, followed by a 6×His tag; HLYAEH: a 7×Ala spacer is attached to the C-terminal end of human lysozyme, followed by a charge-balancing sequence of six negatively charged glutamates (6×Glu), and a 6×His tag).

The human lysozyme variants are constructed with confirmation of correctly engineered sequences. The proteins are expressed and tested for the ability to renature and fold into soluble proteins. Human lysozyme variants are used in pharmaceutical compositions and methods for antimicrobial therapies.

### Experimental Methodology.

Although T4 lysozyme has been successfully expressed and purified using E. coli bacteria, it is difficult to express human lysozyme using this system due partly to the property of human lysozyme's greater bacteria killing activity, and partly to the property of human lysozyme's involving the formation of four disulfide bonds, which is hindered by the reducing environment inside bacteria. At present, the standard practice in human lysozyme expression is to use a yeast expression system, even though it is much less efficient compared to the E. coli system. We have devised an expression and purification strategy that uses an E. coli system to express human lysozyme variants.

Bacterial Expression and purification of the charge-balanced human lysozyme variants. When newly synthesized protein chains are not folded properly, they tend to aggregate and form the bulk of inclusion bodies inside the bacteria, especially when the protein over-expression level is high. This is generally not desired in protein expression since it can produce inactive protein. However, hen egg-white lysozyme can be refolded into active form from a denatured state (Li et al., J. Chrom. A., 2002). We therefore determine whether human lysozyme can be successfully prepared from bacterial expression. Since the reducing bacterial cytosol will probably prevent the correct folding of human lysozme, we expect that when we express our lysozyme variants in bacteria, these proteins will aggregate, form inclusion bodies, and not be active, thus avoiding inhibition of bacterial growth. The inclusion body can be dissolved in denaturing reagents such as 8 M urea solution. In their denatured state, the human lysozyme variants can be immobilized on a cation exchange matrix due to their net positive charge. The immobilized lysozymes are subjected to a gradient of renaturing buffer supplement with redox reagents to encourage disulfide formation. Afterwards the lysozymes can be eluted and again immobilized on nickel-NTA matrix through the 6×His tags, and other proteins from bacteria can be further removed by washing of the matrix. Subsequently the lysozyme variants are eluted from the Ni²⁺-NTA matrix with low pH or imidazole, a histidine analog.

A plasmid containing the human lysozyme gene, Cat. No. TC125273, was purchased from Origene (Rockville, Md.). This plasmid was used as a template for the PCR amplification of the human lysozyme DNA sequence (SEQ ID NO:3). Primers used for constructing HLYH, HLYAH, and HLYAEH (SEQ ID NO:4) are the following, with restriction sequences underlined, and segments relating to FIG. 18.

TABLE 2
Primers for human lysozyme variants.

| | |
|---|---|
| Forward (for all 3 | 5′-tcaccacccatgggaaaggtctttgaaag |
| variants): | gtgtga-3′ |
| (SEQ ID NO:5) | |
| Reverse primer for | 5′-agctacgaagcttagtgatggtgatggtg |
| HLYH: | atgcactccacaaccttga |
| (SEQ ID NO:6) | |
| Reverse primer for | 5′-agctacgaagcttagtgatggtgatggtg |
| HLYAH: | atgtgcggcagctgccgcggcagccactccac |
| (SEQ ID NO:7) | aaccttgaacat-3′ |
| Reverse primer for | 5′-agctacgaagcttagtgatggtgatggtg |
| HLYAEH: | atgctcttcctcttcctcttctgcggcagctg |
| (SEQ ID NO:8) | ccgcggcagccactccacaaccttgaaca |
| | t-3′ |

The PCR products were purified and digested with restriction enzymes Nco I and Hind lIl. The digested and purified DNAs are ligated onto pQE-60 vector (Qiagen, Valencia, Calif.; Cat. No. 32169). E. coli strain M15 with preloaded pREP4 plasmids is transformed with the ligation reaction mixtures. Resulting colonies are sequenced. To express the proteins, transformed M15 cultured were grown in LB, and protein expression was induced by the addition of 1-2 mM IPTG to the culture media.

To purify the proteins, bacteria were harvested with centrifugation and lysed with denaturing buffer A (50 mM Tris, pH 8.7, 8 M urea, 3 mM GSH, 0.3 mM GSSG). The lysates were clarified using ultracentrifugation to remove insoluble materials before loading onto a HiTrap SP XL 1 ml column (GE Healthcare Bio-Sciences, Piscataway, N.J.). A gradient from buffer A to buffer B (100 mM Tris, pH 8.7, 1 M urea, 3 mM GSH, 0.3 mM GSSG) was run through the column using an AKTAFPLC chromatography system (GE Healthcare Bio-Sciences, Piscataway, N.J.). After the refolding, the HiTrap column was directly connected to a HisTrap 1 ml with Ni²⁺-NTA matrix (GE Healthcare Bio-Sciences, Piscataway, N.J.), and the proteins on the cation exchange column were eluted with 300 mM NaCl onto the HisTrap column. The HisTrap column was washed with wash buffer (200 mM NaCl, 50 mM Tris, pH 7.5, and 60 mM imidazole), then eluted with elution buffer (200 mM NaCl, 50 mM Tris, pH 7.5, 200 mM imidazole).

### STATEMENTS REGARDING INCORPORATION BY REFERENCE AND VARIATIONS

All references throughout this application, for example patent documents including issued or granted patents or equivalents; patent application publications; unpublished patent applications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Separate embodiments of the invention are also intended to be encompassed wherein the terms “comprising” or “comprise(s)” or “comprised” are optionally replaced with the terms, analogous in grammar, e.g.; “consisting/consist(s)” or “consisting essentially of/consist(s) essentially of” to thereby describe further embodiments that are not necessarily coextensive.

The invention has been described with reference to various specific and preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be applied to the practice of the invention as broadly disclosed herein without resort to undue experimentation. All art-known functional equivalents of compositions, methods, devices, device elements, materials, procedures and techniques described herein are intended to be encompassed by this invention. Whenever a range is disclosed, all subranges and individual values are intended to be encompassed as if separately set forth. This invention is not to be limited by the embodiments disclosed, including any shown in the drawings or exemplified in the specification, which are given by way of example or illustration and not of limitation. The scope of the invention shall be limited only by the claims.

### REFERENCES

Gronbech-Jensen, N. et al., (1997) “Counterion-induced attraction between rigid polyelectrolytes,” Phys. Rev. Lett. 78:2477-2480.

Lyubartsev, A. P. et al., (1995) “Monte Carlo simulation study of ion distribution and osmotic pressure in hexagonally oriented DNA,” J. Phys. Chem. 99:10373-10382.

Ha, B.-Y. et al., (1997) “Counterion-mediated attraction between two like-charged rods,” Phys. Rev. Lett. 79:1289-1291.

Podgornik, R. et al., (1998) “Charge-fluctuation forces between rodlike polyelectrolytes: Pairwise summability reexamined,” Phys. Rev. Lett. 80:1560-1563.

Stevens, M. J., (1999) “Bundle binding in polyelectrolyte solutions,” Phys. Rev. Lett. 82:101-104.

Rouzina, I. et al., (1996) “Macroion attraction due to electrostatic correlation between screening counterions. 1. Mobile surface-adsorbed ions and diffuse ion cloud,” J. Phys. Chem. 100:9977-9989.

Shklovskii, B. I. (1999) “Wigner crystal model of counterion induced bundle formation of rodlike polyelectrolytes,” Phys. Rev. Lett. 82:3268-3271.

Manning, G. S. (1978) “The molecular theory of polyelectolyte solutions with applications to the electrostatic properties of polynucleotides.” Q. Rev. Biophys. 2:179-246.

Angelini, T. et al., (2003) “Like-charge attraction between polyelectrolytes mediated by counterion charge density waves,” Proc. Nat. Acad. Sci. USA 100:8634-8637.

Raedler, J. O. et al., (1997) “Structure of DNA-cationic liposome complexes: DNA intercalation in multi-lamellar membranes in distinct interhelical packing regimes,” Science 275:810-813.

Wong, G. C. L. et al., (2003) “A lamellar phase of stacked two-dimensional rafts of actin filaments,” Phys. Rev. Lett. 91:018103.

Butler, J. et al., (2003) Ion multivalence and like-charge attraction,” Phys. Rev. Lett. 91:028301.

Sheils, C. A. et al., (1996) “Actin filaments mediate DNA fiber formation in chronic inflammatory airway disease,” Am. J. Pathol. 148:919-927.

Lam, J. et al., (1980) “Production of mucoid microcolonies by Pseudomonas aeroginosa within infected lungs in cystic fibrosis,” Infect. Immun. 28:546-6.

Lewis, R. W. (1978) “The biochemical basis of cystic fibrosis: A hypothesis based upon the polyelectrolytes of mucus,” Tex. Rep. Bio. Med. 36:33-38.

Vasconcellos, C. A. et al., (1994) “Reduction in viscosity of cystic fibrosis sputum in vitro by gelsolin,” Science 263:969-971.

Brandt, T. et al., (1995) “DNA concentration and length in sputum of patients with cystic fibrosis during inhalation with recombinant human Dnase,” Thorax 50:880-882.

Shak, S. et al., (1990) “Recombinant human Dnase I reduces the viscosity of cystic fibrosis sputum,” Proc. Nat. Acad. Sci. USA 95:14961-14966.

Travis, S. M. et al., (1999) “Activity of abundant antimicrobials of the human airway,” Am. J. Respir. Cell. Mol. Biol. 20:872-879.

Zabner, J. et al., (2000) “The osmolyte xylitol reduces the salt concentration of airway surface liquid and may enhance bacterial killing,” Proc. Nat. Acad. Sci. USA 97:11615-11619.

Weiner, D. J. et al., (2003) “The antimicrobial activity of cathelicidin LL37 is inhibited by F-actin bundles and restored by gelsolin,” Am. J. Res. Cell. Mol. Bio. 28:738-745.

Sanders, L. K. et al., (2005) “Structure and stability of self-assembled actin-lysozyme complexes in salty water,” Phys. Rev. Lett. 95(10):108302.

Guaqueta, C. et al., (2006) “The Effect of Salt on Self-Assembled Actin-Lysozyme Complexes”, Biophys. J. 90:4630-4638.

S. Dao-pin et al., (1991) “Cumulative site-directed charge-change replacements in bacteriophage T4 lysozyme suggest that long-range electrostatic interactions contribute little to protein stability,” J. Mol. Biol. 221:873-887.

Tang J X, Wen Q, Bennett A, Kim B, Sheils C A, Bucki R, Janmey P A; Anionic poly(amino acid)s dissolve F-actin and DNA bundles, enhance DNase activity, and reduce the viscosity of cystic fibrosis sputum; Am J Physiol Lung Cell Mol Physiol. 2005 Oct;289(4):L599-605.

Gibson, R. L. et al., (2003) “Pathophysiology and management of pulmonary infections in cystic fibrosis,” Am. J. Respir. Crit. Care Med. 168:918-915.

Mendelman, P. M. et al., (1985) “Aminoglycoside penetration, inactivation and efficacy in cystic fibrosis sputum,” Am. Rev. Respir. Dis. 132:761-765.

Hunt, B. E. et al., (1995) “Macromolecular mechanisms of sputum inhibition of tobramycin activity,” Antimicrob. Agents Chemother. 39:34-39.

Rampal, R. et al., (1988) “The binding of anti-pseudomonal antibiotics to macromolecules from cystic fibrosis sputum,” J. Antimicrob. Chemother. 33:483-490.

Zribi, O. V. et al. (2005) “Salt-induced condensation in actin-DNA mixtures,” Europhys. Lett. 70:541-547.

Koltover, I. et al., (1998) “An inverted hexagonal phase of cationic liposome-DNA complexes related to DNA release and delivery,” Science 281:78-81.

Ewert, K. et al., (2004) “Cationic lipid-DNA complexes for gene therapy: Understanding the relationship between complex structure and gene delivery pathways at the molecular level,” Current Medicinal Chemistry 11:133-149.

Janmey, P. A. et al., (1986) J. Biol. Chem. 261 8357-62.

Rommens, R. J. et al., (1989) “Identification of the cystic fibrosis gene: chromosome walking and jumping,” Science 245,1059-1065.

Riordan, J. R., et al., (1989) “Identification of the cystic fibrosis gene: cloning and characterization of complementary DNA,” Science 245,1066-1073.

Kerem, B., et al., (1989) “Identification of the cystic fibrosis gene: genetic analysis,” Science 245,1073-1080.

Welsh, M. J., and Smith, A., (1995) “Cystic fibrosis,” Scientific American 274, 52-59.

Bals, R.,et al., (1998) “Mouse beta defensin 1 is a salt sensitive antimicrobial peptide present in epithelia of the lung and urogenital tract” Infect Immun. 66,1225-32.

Bals, R., et al., (1998) “Human beta defensin 2 is a salt sensitive peptide antibiotic expressed in human lung” J. Clin. Invest. 102, 874-80.

Smith, J. J., et al., (1996) “Cystic fibrosis airway epithelia fail to kill bacteria because of abnormal airway surface liquid” Cell 85, 229-36.

Weinberg, M., et al., (1988) “Inhibition of human neutrophil elastase by bacterial polyanions” Exp. Lung Res. 14, 67-83

Singh, P. K., et al. (1998) “Production of beta defensins by human airway epithelia” Proc. Nat. Acad. Sci. USA. 95,14961-6.

Vered, M., et al., (1988) “Inhibition of human neutrophil elastase by bacterial polyanions” Exp. Lung Res. 14, 67-83.

Kirkwood, J. G. and J. B. Shumaker (1952) Proc. Nat. Acad. Sci. U.S.A. 38, 863-871.

Oosawa, F. (1968) “Interactions between parallel rodlike macroions”, Biopolymers 6,1633-1647.

Podgornik, R and V. A. Parsegian (1998), “Charge-fluctuation forces between rodlike polyelectrolytes: Pairwise summability reexamined”, Phys. Rev. Lett. 80,1560-1563.

Kornyshev, A.A. and S. Leikin, (1999) “Electrostatic zipper motif for DNA aggregation”, Phys. Rev. Lett. 82, 4138-4141

Angelini, T., et al. (2003) “Like-charge attraction between polyelectrolytes mediated by counterion charge density waves”, Proc. Nat. Acad. Sci. USA 100, 8634-8637.

Felgner, P. L. et al., (1987) “Lipofection: a highly efficient, lipid-mediated DNA transfection procedure”, Proc. Nat. Acad. Sci. USA 84, 7413-7417.

Felgner, P. L. and G. Rhodes, (1991) “Gene Therapeutics”, Nature 349, 351-352.

Gustafsson, J. et al., (1995) “Complexes between cationic liposomes and DNA visualized by cryo-TEM”, Biochim. Biophys. Acta-Biomem. 1235, 305-312.

Sternberg, B. et al., (1994) “New structures in complex formation between DNA and cationic liposomes visualized by freeze-fracture electron microscopy”, FEBS Lett. 356, 361-366.

Lasic, D. D. et al., (1997) “The structure of DNA-liposome complexes”, J. Am. Chem. Soc. 119, 832-833.

May, S. and A. Ben-Shaul, (1997) “DNA-lipid complexes: stability of honeycomb-like and spaghetti-like structures”, Biophys. J. 73, 2427-2440.

Bruinsma, R. (1998) “Electrostatics of DNA cationic lipid complexes: isoelectric instability”, European. Phys. J. B 4, 75-88.

Harries, D. et al., (1998) “Structure, stability, and thermodynamics of lamellar DNA-lipid complexes”, Biophys. J. 75,159-173.

Wong, G. C. L. et al., (2000) “Hierarchical self-Assembly of F-actin cationic lipid complexes: stacked three-layer tubule networks,” Science 288, 2035-2039.

Expression system kind gift from Prof. Brian W. Matthews, University of Oregon.

Citron, D. M., et al., (1994) in Bailey & Scott's Diagnostic Microbiology, eds. Baron, E. J., Peterson, L. R. & Finegold, S. M. (Mosby, St. Louis), pp. 219-233.

Woods, G. L. and J. A. Washington (1995) in Mandell, Douglas and Bennet's Principles and Practice of Infectious Diseases, eds. Mandell, G. L., Bennett, J. E. & Dolin, R. (Churchill Livingstone, New York), Vol. 1, pp. 169-175.

Zhou S., et al., (2004) “Nanostructures of Complexes Formed by Calf Thymus DNA Interacting with Cationic Surfactants”, Biomacromolecules 5, 1256-1261.

Song, H., et al., (1994) “Structural changes of active site cleft and different saccharide binding modes in human lysozyme co-crystallized with hexa-N-acetyl-chitohexaose at pH 4.0”; J. Mol. Biol. 244:522-540.

Li M., et al., (2002) “Dual gradient ion-exchange chromatography improved refolding yield of lysozyme.” J. Chrom. A, 959,113-120.

1. A charge-modified antimicrobial lysozyme, wherein the charge-modified lysozyme is a derivative of a reference lysozyme protein and has a reduction of a net charge relative to the reference lysozyme protein net charge.

2. The charge-modified lysozyme of claim 1, wherein said reference lysozyme is a mammalian lysozyme.

3. The charge-modified lysozyme of claim 1, wherein said reference lysozyme is a human lysozyme.

4. The charge-modified lysozyme of claim 1, wherein the reduction of the net charge is an amount selected from the group consisting of about 2, about 3, about 4, about 5, about 6, about 7, and about 8.

5. The charge-modified lysozyme of claim 1, wherein the reduction of the net charge is at least about two.

6. The charge-modified lysozyme of claim 1, wherein the reduction of the net charge level is at least about four.

7. The charge-modified lysozyme of claim 1, wherein the charge-modified lysozyme has a relative antimicrobial activity selected from the group consisting of at least about 20%, at least about 50%, at least about 60%, at least about 70%, at least about 80%, and at least about 90% in comparison with a reference antimicrobial activity of the reference lysozyme protein.

8. The charge-modified lysozyme of claim 1, wherein the charge-modified lysozyme has a charge-modified antimicrobial activity of at least about 50% in comparison with a reference antimicrobial activity of the reference lysozyme protein.

9. A method of potentiating an antimicrobial activity of a lysozyme, comprising modifying a net charge level of the lysozyme.

10. The method of claim 9 wherein said modifying is by reducing a net charge level to a less positive net charge level.

11. A method of treating a microbial infection, comprising administering to a patient in need the composition of claim 1.

12. The method of claim 11, wherein said administering is by aerosol delivery.

13. The method of claim 11, wherein said administering is to an upper respiratory tract region.

14. The method of claim 11, wherein the patient is a cystic fibrosis patient.

15. A method of generating a non-stick, charge-modified form of an antimicrobial protein, comprising the steps of: (a) providing a candidate antimicrobial protein or sequence information corresponding to nucleic acids or amino acids thereof; (b) developing at least one charge-modified version of said candidate antimicrobial protein; (c) screening said charge-modified version for antimicrobial activity; and (d) selecting an active charge-modified version; thereby generating said non-stick, charge-modified form of an antimicrobial protein.

16. A method of potentiating an antibiotic treatment, comprising the steps of (a) administering a surfactant composition; and (b) administering the antibiotic to a patient in need of treatment.

17. The method of claim 16 wherein the surfactant composition is at least partially cationic.

18. The method of claim 16 wherein the surfactant composition is a cationic lipid composition.

19. The method of claim 16 wherein the surfactant composition is selected from the group consisting of: Didodecyldimethylammonium bromide (DDAB);
Cetyltrimethylammonium bromide (CTAB); Cetyltrimethylammonium bromide (CTAB); 1-palmitoyl-2-hydroxy-sn-glycero-3-phosphocholine (PHGPC) from 20:80 to 80:20; DLTAP:DLPC, DOTAP:DOPC, and DNTAP:DNPC (dilauryl trimethyl ammonium propane: dilauryl trimethyl phosphatidylcholine, dioleoyl trimethyl ammonium propane: dioleoyl trimethyl phosphatidylcholine, and dinervonyl trimethyl ammonium propane: dinervonyl trimethyl phosphatidylcholine, respectively) from 100:0 to 10:90.

20. The method of claim 16 wherein the surfactant composition is selected from the group consisting of: 1,2-Diarachidonoyl-sn-Glycero-3-Phosphoethanolamine; 1,2-Didocosahexaenoyl-sn-Glycero-3-Phosphoethanolamine; 1,2-Dielaidoyl-sn-Glycero-3-Phosphoethanolamine; 1,2-Dihexanoyl-sn-Glycero-3-Phosphoethanolamine; 1,2-Dioctanoyl-sn-Glycero-3-Phosphoethanolamine; 1,2-Dihexanoyl-sn-Glycero-3-Phosphoethanolamine; 1,2-Dilauroyl -sn-Glycero-3-Phosphoethanolamine; 1,2-Dimyristoyl-sn-Glycero-3-Phosphoethanolamine; 1,2-Dipalmitoyl-sn-Glycero-3-Phosphoethanolamine; 1,2-Dipalmitoleoyl -sn-Glycero-3-Phosphoethanolamine; 1,2-Diheptadecanoyl-sn-Glycero-3-Phosphoethanolamine; 1,2-Dicapryl-sn-Glycero-3-Phosphoethanolamine; 1,2-Dimyristoyl-3-Trimethylammonium-Propane; 1,2-Dipalmitoyl-3-Trimethylammonium-Propane; 1,2-Dimyristoleoyl-sn-Glycero-3-Phosphocholine; 1,2-Dimyristelaidoyl-sn-Glycero-3-Phosphocholine; 1,2-Dipalmitoleoyl-sn-Glycero-3-Phosphocholine; 1,2-Dipalmitelaidoyl-sn-Glycero-3-Phosphocholine; 1,2-Dieicosenoyl-sn-Glycero-3-Phosphocholine; 1,2-Dierucoyl-sn-Glycero-3-Phosphocholine; 1,2-Dinervonoyl-sn-Glycero-3-Phosphocholine; 1,2-Dipetroselinoyl-sn-Glycero-3-Phosphocholine; and 1,2-Dielaidoyl-sn-Glycero-3-Phosphocholine.

21. The method of claim 16 wherein the surfactant composition comprises DOTAP and DOPE.

22. The method of claim 21 wherein the surfactant composition has a DOTAP:DOPE ratio of from about 100:0 to about 10:90.

23. The method of claim 22 wherein the surfactant composition has a DOTAP:DOPE ratio of from about 70:30 to about 25:75.

24. The method of claim 16 wherein the antibiotic is a positively-charged aminoglycoside antibiotic.

25. The method of claim 16 wherein the antibiotic is selected from the group consisting of: tobramycin, gentamycin, kanamycin, streptomycin, neomycin, amikacin, and ampramycin.

26. The method of claim 16 wherein the antibiotic is tobramycin.

27. The method of claim 16 wherein the patient is a cystic fibrosis patient.

28. A method of generating a positively-charged surfactant formulation for therapeutic use in connection with a positively-charged antimicrobial agent, wherein said therapeutic use involves an electrostatic environment with at least one anionic component, comprising: (a) identifying said at least one anionic component; (b) providing a positively charged surfactant formulation candidate; (c) maximizing an entropic gain of said candidate upon binding said anionic component by optimizing one or more of charge density and surfactant curvature of said surfactant formulation candidate; and (d) selecting a formulation candidate exhibiting an entropy gain from said maximizing step; thereby generating a positively-charged surfactant formulation for therapeutic use in connection with the positively-charged antimicrobial agent.

29. A charge-modified antimicrobial lysozyme, wherein the charge-modified lysozyme is a derivative of a reference lysozyme protein and has one or more charge decreases relative to the reference lysozyme protein.

30. The charge-modified lysozyme of claim 1, excepting a mutant T4 bacteriophage lysozyme as described herein and those other lysozymes which may be known in the art that do qualify as prior art.

31. A method of potentiating an antibiotic treatment, comprising the steps of (a) administering an amphiphilic molecule composition; and (b) administering the antibiotic to a patient in need of treatment.

32. The method of claim 31 wherein said amphiphilic molecule is at least partially cationic.

33. A method of generating an amphiphilic molecule formulation for therapeutic use in connection with a positively-charged antimicrobial agent, wherein said therapeutic use involves an electrostatic environment with at least one anionic component, comprising: (a) identifying said at least one anionic component; (b) providing an amphiphilic molecule formulation candidate; (c) maximizing an entropic gain of said candidate upon binding said anionic component by optimizing one or more of charge density and curvature of said candidate; and (d) selecting a formulation candidate exhibiting an entropy gain from said maximizing step; thereby generating an amphiphilic molecule formulation for therapeutic use in connection with the positively-charged antimicrobial agent.

34. A charge-modified mammalian lysozyme comprising a first segment having of an amino acid sequence of a mammalian lysozyme, and a second segment having from about two to about ten negatively charged amino acids.

35. The charge-modified mammalian lysozyme of claim 34 wherein the second segment comprises six negatively charged amino acids.

36. The charge-modified mammalian lysozyme of claim 34 wherein the second segment comprises six glutamate residues.

37. The charge-modified mammalian lysozyme of claim 34 further comprising a third segment of a spacer, wherein said spacer is positioned between said first segment and said second segment.

38. The charge-modified mammalian lysozyme of claim 37 wherein the spacer is a peptide comprising from about two to about ten amino acids.

39. The charge-modified mammalian lysozyme of claim 37 wherein the spacer comprises seven alanine residues.

40. The charge-modified mammalian lysozyme of claim 34 wherein the lysozyme is human.

41. The charge-modified mammalian lysozyme of claim 34 having the amino acid sequence of SEQ ID NO:4.

42. A nucleic acid sequence capable of encoding a charge-modified lysozyme.

43. A method of treating an infection condition involving a prolonged inflammatory response, comprising administering to a patient in need the composition of claim 1.

44. The method of claim 43 wherein the infection is a chronic microbial infection.
